(19) United States
(12) Patent Application Publication
(10) Pub. No.: US 2026/0117203 A1
(43) Pub. Date: Apr. 30, 2026

(54) MUTANT KETOREDUCTASE WITH INCREASED KETOREDUCTASE ACTIVITY AS WELL AS METHODS AND USES INVOLVING THE SAME

(71) Applicant: Hoffmann-La Roche Inc., Little Falls, NJ (US)

(72) Inventors: Rebecca Maria Ursula Buller, Wädenswil (CH); Nadine Nina Duss, Wädenswil (CH); Steven Paul Hanlon, Basel (CH); Sumire Honda Malca, Wädenswil (CH); Hans Iding, Basel (CH); Bernd Willi Kuhn, Basel (CH); Jasmin Claudia Meierhofer, Wädenswil (CH); Michael Niklaus, Wädenswil (CH); David Patsch, Wädenswil (CH)

(21) Appl. No.: 19/286,007

(22) Filed: Jul. 30, 2025

Related U.S. Application Data
(63) Continuation of application No. PCT/EP2024/052267, filed on Jan. 30, 2024.

(30) Foreign Application Priority Data
Jan. 31, 2023 (EP) 23154331.5

(51) Int. Cl. C12N 9/04; C12P 17/16

(52) U.S. Cl. CPC C12N 9/0006; C12P 17/165

(57) ABSTRACT

The present invention relates to a mutant ketoreductase with at least one mutation at position 241, a nucleic acid encoding the mutant ketoreductase, a vector comprising the nucleic acid, a method for the enzymatic reduction of a ketone and the formation of a chiral alcohol with the mutant ketoreductase, the use of the mutant ketoreductase for the reduction of ketones and the formation of chiral alcohols as well as the use of the method for the preparation of pharmaceutically active serine/threonine protein kinase inhibitors.

### CROSS-REFERENCE TO RELATED APPLICATIONS

This application is a continuation of International PCT Application No. PCT/EP2024/052267 filed on Jan. 30, 2024, which claims priority to European Patent Application No. 23154331.5 filed on Jan. 31, 2023, the contents of each application are incorporated herein by reference in their entireties.

### SEQUENCE LISTING

This application incorporates by reference the material in the ST.26 XML file titled P37709-US-1_Sequence-Listing_ROCHE-242, which was created on Jul. 30, 2025 and is 11,888 bytes.

The present invention relates to a mutant ketoreductase with at least one mutation at position 241, a nucleic acid encoding the mutant ketoreductase, a vector comprising the nucleic acid, a method for the enzymatic reduction of a ketone and the formation of a chiral alcohol with the mutant ketoreductase as well as the use of the mutant ketoreductase for the preparation of pharmaceutically active serine/threonine protein kinase inhibitors.

Ketoreductases are a subclass of enzymes belonging to the group of oxidoreductases, i.e., enzymes catalyzing redox reactions allowing the transfer of an electron from a so-called electron donor molecule to an electron acceptor molecule.

The subclass of ketoreductases have the specific capability of catalyzing the asymmetric reduction of desired ketones to their corresponding secondary alcohol.

During this reduction reaction, electrons are transferred to the ketone group (C═O) by the addition of a hydride to the carbonyl atom of the ketogroup. In addition, a proton is transferred to the oxygen of the carbonyl group. This reaction generally requires hydride donors as cofactors, such as NADH or NADPH, which may be regenerated in-situ and protonating amino acids residues in the active site of the ketoreductases.

By now, ketoreductases are also commonly used in the enzymatic reduction of prochiral keto compounds and accordingly for the preparation of intermediates for various pharmaceutical compounds, such as for instance in the preparation of serine/threonine protein kinase inhibitors of the formula

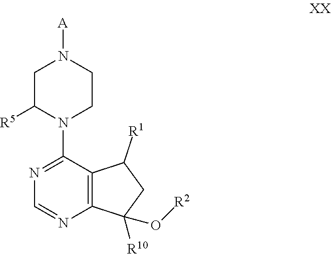

as illustrated e.g. in the PCT International Application WO 2008/006040 A1. The protein kinase inhibitors are useful for example for the treatment of hyperproliferative diseases, such as cancer and inflammation in mammals.

A particular promising serine/threonine protein kinase inhibitor is the clinical AKT inhibitor candidate ipatasertib (CAS Reg. No. 1001264-89-6), which has the formula

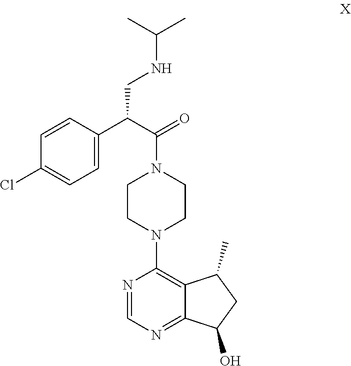 [SMILES: CC(C)NC[C@H](C(=O)N1CCN(c2ncnc3c2[C@H](C)C[C@H]3O)CC1)c1ccc(Cl)cc1]

It is an object of the present invention to design improved mutant ketoreductases with an increased ketoreductase activity relative to the wildtype ketoreductase, particularly the ketoreductase of Sporidiobolus salmonicolor, especially that of SEQ ID NO: 1. These mutants may be used in the production of chiral alcohols, such as chiral alcohols of formula I, including its production in a scaled-up process.

Surprisingly, it has been found that a mutant ketoreductase comprising an amino acid sequence that is at least 80% identical to the amino acid sequence of SEQ ID NO: 1 (ketoreductase from Sporidiobolus salmonicolor; referred to as Q9UUN9 in UniProt) and has at least one amino acid substitution relative to the amino acid sequence of SEQ ID NO: 1, wherein the amino acid at the position corresponding to position 241 of SEQ ID NO: 1 is substituted, shows an increased ketoreductase activity relative to the wildtype ketoreductase, particularly the ketoreductase of Sporidiobolus salmonicolor, especially that of SEQ ID NO: 1.

As shown in the Examples, substitutions introduced at position 241 of the wild-type ketoreductase from Sporidiobolus salmonicolor as defined in SEQ ID NO: 1 (referred to as Q9UUN9 in UniProt) confer increased activity to the mutant relative to the wild-type enzyme. As shown in Table 1, substitutions of leucine at position 241 with Met, Asn, Arg, Trp, Ile or Lys increased the ketoreductase activity. Additionally, increase in activity could be proven for further substitutions, particularly with Ala, Cys, Tyr, Val, Gln, Phe, His, Gly, Asp, Ser and Thr, when combined with substitutions at other sites, namely at positions 97, 174, 238, 242, and/or 245 (see Table 2) or particularly 97, 241 and 245 (see Table 4). Substitutions at positions 242 and 245 were shown to be of particular relevance in further increasing the enzyme activity (see Tables 4 to 7), which can be further increased by substituting Phe at position 97 with Trp (see Tables 8 and 9). The inventors identified still further substitutions which can further assist in increasing ketoreductase activity, namely those at positions 134, 174, 224, 228, 234, 238, 246, 316 and/or 342 (see Tables 10 to 20). It could be confirmed that increased enzyme activity is present at different time points, temperatures and co-substrate concentrations (see Tables 22 to 24). High diastereoselectivity of the wild-type enzyme is maintained in the mutants at different scales and with different recycling systems (see Tables 25 to 27). Reductase performance and diastereoselectivity was confirmed for ketoreductase form different fungal species (see Table 21).

In addition, it has been found that such ketoreductase are highly active for catalysing the enzymatic reduction of ketones and for the formation of chiral alcohols.

Moreover, it has been found that the mutant ketoreductase of the present invention in comparison to the wild-type ketoreductase show an increased conversion in the enzymatic reduction of ketones.

Especially it has been found that the enzymatic reduction of a ketone of the formula

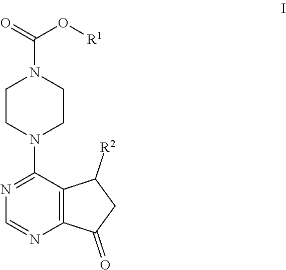

wherein R¹ is C₁₋₄-alkyl and R² is hydrogen or C₁₋₄-alkyl, particularly methyl or ethyl, especially methyl
with the mutant ketoreductase of the present invention forms the chiral alcohol of the formula II

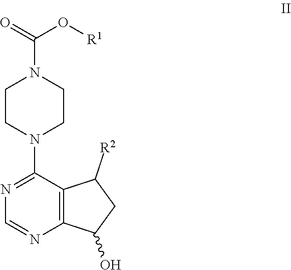

wherein R¹ and R² are as above, with a high enantiomeric or respectively diastereomeric excess. Therefore, the ketoreductase according to the present invention is extremely useful for the preparation of chiral alcohol key intermediates, particularly for key intermediates in the preparation of serine/threonine protein kinase inhibitors of the formula

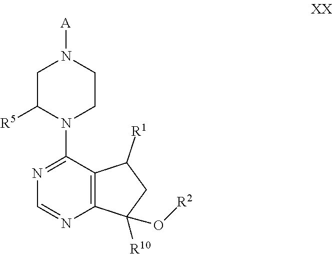

as illustrated e.g. in the PCT International Application WO 2008/006040 A1.

Accordingly, in a first aspect the present invention relates to a mutant ketoreductase with increased ketoreductase activity relative to the wild-type ketoreductase, wherein the mutant ketoreductase comprises an amino acid sequence that is at least 80% identical to the amino acid sequence of SEQ ID NO: 1 (ketoreductase from Sporidiobolus salmonicolor; UniProt ID: Q9UUN9); and wherein the mutant ketoreductase has at least one amino acid substitution relative to the amino acid sequence of SEQ ID NO: 1, wherein the amino acid at the position corresponding to position 241 of SEQ ID NO: 1 is substituted.

The term “ketoreductase” as used herein means any protein having the capability of asymmetrically catalyzing the reduction of a ketone to the corresponding chiral, non-racemic secondary alcohol, particularly the pure enantiomers respectively diastereomers of a secondary alcohol.

The term “ketone” as used herein means a substrate with a prochiral keto functionality, which may comprise further chiral centers.

The term C₁₋₄-alkyl as used herein means for the R¹ substituent a monovalent linear or branched saturated hydrocarbon group of 1 to 4 carbon atoms such as methyl, ethyl, n-propyl, isopropyl, n-butyl, i-butyl, sec-butyl, or t-butyl, preferably t-butyl.

The term C₁₋₄-alkyl as used herein means for the R² substituent a monovalent linear saturated hydrocarbon group of 1 to 4 carbon atoms, such as methyl, ethyl, n-propyl, or n-butyl, preferably methyl.

The term “wild-type ketoreductase” as used herein means any ketoreductase which occurs as such in nature. The term “mutant ketoreductase” as used herein means any ketoreductase, which originates from a corresponding wild-type ketoreductase and in comparison to such wild-type ketoreductase has been amended in its amino acid sequence. For example, this may comprise the introduction, deletion, substitution or post-translational mutation of one or more amino acids at one or more positions. Preferably, the mutant ketoreductase differs from the wild-type ketoreductase by amino acid substitutions. Methods for creating mutations, such as amino acid substitutions, in amino acid sequences are well-known to the person skilled in the art. For example, such mutations may already be introduced on nucleic acid level leading to the expression of the desired mutated amino acid sequence. Suitable methods therefore are well-known to the person skilled in the art and partly also described below, e.g. in the context of nucleic acids according to the second aspect of the invention.

Suitable mutant ketoreductases according to the first aspect may originate from the wild-type ketoreductase of any organism. Proven activity has been found using wild type ketoreductases form organisms such as Scheffersomyces stipitis, Clavispora lusitaniae, Meyerozyma guilliermondii, Tilletiopsis washingtonensis, Rachicladosporium antarcticum, Lodderomyces elongisporus, Acidomyces richmondensis, or Plicaturopsis crispa. Especially preferred is the Sporidiobolus salmonicolor ketoreductase, referred to as Q9UUN9 in UniProt.

Also disclosed in the present application are mutants of wild-type ketoreductase of organisms other than Sporidiobolus salmonicolor, in which any of the mutations or combinations thereof identified for Sporidiobolus salmonicolor and/or defined in the present application may be introduced at the corresponding position(s) of the other organisms. The organism may be, e.g., Scheffersomyces stipitis, Clavispora lusitaniae, Meyerozyma guilliermondii, Tilletiopsis washingtonensis, Rachicladosporium antarcticum, Lodderomyces elongisporus, Acidomyces richmondensis or Plicaturopsis crispa. Corresponding positions may be identified by e.g. sequence alignment and identification of homologous region as known to the skilled practitioner. Reductase performance and diastereoselectivity of ketoreductases from different fungal species in comparison to the wild-type ketoreductase from Sporidiobolus salmonicolor (SEQ ID NO: 1; UniProt ID: Q9UUN9) are shown in Table 21.

In accordance with the present invention, the mutant ketoreductase is active as ketoreductase. This means that the mutant ketoreductase is capable of converting a prochiral ketone to the corresponding secondary alcohol under suitable conditions, as detailed above and below. Methods for determining ketoreductase activity are described herein and given in the Examples.

The mutant ketoreductase according to the first aspect shows increased ketoreductase activity relative to the wild-type ketoreductase.

The activity may be determined in an enzyme assay measuring either the consumption of substrate or production of product over time. A large number of different methods of measuring the concentrations of substrates and products exist and many enzymes can be assayed in several different ways as known to the person skilled in the art.

Methods of determining enzymatic activity of a mutant ketoreductase according to the present invention or a wild-type ketoreductase are well-known to the person skilled in the art. Exemplary methods are also described in the Examples. To determine, whether a mutant ketoreductase according to the first aspect shows increased ketoreductase activity relative to the wild-type ketoreductase, ketoreductase activity of both ketoreductases is measured using the same method.

For example, methods of determining enzymatic activity of a ketoreductase in general may be based on a fluorescence or colorimetric assay. Further, methods of determining enzymatic activity of a ketoreductase in general may comprise the detection of the concentration of product being formed, the substrate consumed or the detection of formed or consumed cofactors necessary for the reaction, such as the concentrations of NAD⁺, NADH, NADP⁺ or NADPH.

A mutant ketoreductase according to the first aspect showing increased ketoreductase activity relative to the wild-type ketoreductase, for example, shows an increase in ketoreductase activity by more than the onefold. The person skilled in the art knows statistical procedures to assess whether or not one value of enzyme activity is increased relative to another, such as Student's t-test or chi-square test. It is evident for the skilled person that any background signal has to be subtracted when analyzing the data.

Further, the mutant ketoreductase according to the first aspect of the invention comprises an amino acid sequence that is at least 80% identical to the amino acid sequence of SEQ ID NO: 1 (Sporidiobolus salmonicolor ketoreductase, referred to as Q9UUN9 in UniProt). Thereby, the amino acid sequence of SEQ ID NO: 1 originates from Sporidiobolus salmonicolor ketoreductase, which is referred to as Q9UUN9 in UniProtKB.

The term “sequence identity” as used herein describes the percentage of characters that exactly match between two different sequences.

For example, the term “at least 80% identical to the amino acid sequence of SEQ ID NO: 1” as used herein means that the amino acid sequence of the mutant ketoreductase of the present invention has an amino acid sequence characterized in that, within a stretch of 100 amino acids, at least 80 amino acid residues are identical to the sequence of the corresponding sequence of SEQ ID NO: 1.

The mutant ketoreductase may also comprise an amino acid sequence that is at least 85%, 90%, 95%, 96%, 97%, 98% or 99% identical to the amino acid sequence of SEQ ID NO: 1 (Sporidiobolus salmonicolor ketoreductase, referred to as Q9UUN9 in UniProt).

Sequence identity according to the present invention can, e.g., be determined by methods of sequence alignment in form of sequence comparison. Methods of sequence alignment are well known in the art and include various programs and alignment algorithms. Moreover, the NCBI Basic Local Alignment Search Tool (BLAST) is available from several sources, including the National Center for Biotechnology Information (NCBI, Bethesda, MD) and on the internet, for use in connection with the sequence analysis programs blastp, blastn, blastx, tblastn and tblastx. Percentage of identity of mutants according to the present invention relative to the amino acid sequence of e.g. SEQ ID NO: 1 is typically characterized using the NCBI Blast blastp with standard settings. Alternatively, sequence identity may be determined using the software GENEious with standard settings. Alignment results can be, e.g., derived from the Software CLC Main Workbench (version 21), using the global alignment protocol with free end gaps as alignment type, and Blosum62 as a cost matrix.

The mutant ketoreductase according to the present invention has at least one amino acid substitutions relative to the amino acid sequence of SEQ ID NO: 1. Moreover, the mutant ketoreductase according to the present invention may have at least two, three, four, five, six, seven, eight, nine, ten or more amino acid substitutions relative to the amino acid sequence of SEQ ID NO: 1.

Especially, the mutant ketoreductase according to the present invention has at least one amino acid substitutions relative to the amino acid sequence of SEQ ID NO: 1, wherein the amino acid at the position corresponding to position 241 of SEQ ID NO: 1 is substituted.

Methods for preparing the mutant ketoreductase according to the first aspect of the present invention are well-known to the person skilled in the art. For example, the mutant ketoreductase according to the first aspect may be prepared by using any method suitable for preparing a recombinant enzyme known to the person skilled in the art, such as recombinant expression of the modified nucleic acid of the mutant ketoreductase in cell culture, followed by protein isolation and purification.

Preferably, in the mutant ketoreductase of the first aspect, wherein the amino acid at the position corresponding to position 241 of SEQ ID NO: 1 is substituted with Met (Met241), Asn (Asn241), Arg (Arg241), Trp (Trp241), Ile (241Ile), Lys (Lys241), His (His241), Gln (Gln241), Gly (Gly241), Asp (Asp241), Ser (Ser241), Thr (Thr241), Tyr (Tyr241), Cys (Cys241), Ala (Ala241), Val (Val241), or Phe (Phe241).

Even more preferred, in the mutant ketoreductase of the first aspect, the amino acid at the position corresponding to position 241 of SEQ ID NO: 1 is substituted with Met (Met241), Gln (Gln241), Cys (Cys241), Tyr (Tyr241), Ser (Ser241), Thr (Thr241), Val (Val241), or Ala (Ala241), more preferably Met (Met241).

If the mutant ketoreductase according to the first aspect has more than one amino acid substitution relative to the amino acid sequence of SEQ ID NO: 1, such as two, three, four, five, six, seven, eight, nine, ten or more amino acid substitutions relative to the amino acid sequence of SEQ ID NO: 1, the amino acid sequence of the mutant ketoreductase of the first aspect of the invention preferably comprises substitutions at positions corresponding to positions 242 and/or 245, of SEQ ID NO: 1 in addition to that at position 241.

Accordingly, in a preferred embodiment of the mutant ketoreductase of the first aspect has at least substitutions at the following positions:
241 and 242; or
241 and 245; or
241, 242 and 245.

In a more preferred embodiment of the mutant ketoreductase of the first aspect has at least the following substitutions:
the amino acid at the position corresponding to position 241 of SEQ ID NO: 1 is substituted with Met (Met241), Asn (Asn241), Arg (Arg241), Trp (Trp241), Ile (241Ile), Lys (Lys241), His (His241), Gln (Gln241), Gly (Gly241), Asp (Asp241), Ser (Ser 241), Thr (Thr241), Tyr (Tyr241), Cys (Cys241), Ala (Ala241), Val (Val241), or Phe (Phe241), preferably Met (Met241), Gln (Gln241), Cys (Cys241), Tyr (Tyr241), Ser (Ser 241), Thr (Thr241), Val (Val241), or Ala (Ala241), more preferably Met (Met241); and/or
the amino acid at the position corresponding to position 242 of SEQ ID NO: 1 is substituted with Trp (Trp242), Phe (Phe242), Ile (Ile242), Tyr (Tyr242); Cys (Cys242); Val (Val242), Leu (Leu242), Pro (Pro242), Ala (Ala242), Gln (Gln242) or Ser (Ser242), preferably Trp (Trp242), Phe (Phe242), Ile (Ile242), or Tyr (Tyr242), more preferably Trp (Trp242) or Ile (Ile242), most preferably Trp (Trp242); and/or
the amino acid at the position corresponding to position 245 of SEQ ID NO: 1 is substituted with Ser (Ser245), Thr (Thr245), Asn (Asn245), Met (Met245), Asp (Asp245), Trp (Trp245), Phe (Phe245), Glu (Glu245), Cys (Cys245), or His (His245), preferably Ser (Ser245), Thr (Thr245), or Asn (Asn245), more preferably Ser (Ser245) or Thr (Thr245), most preferably Ser (Ser245).

More preferably, the mutant ketoreductase of the present invention is characterized in that
the amino acid at the position corresponding to position 241 of SEQ ID NO: 1 is substituted with Met (Met241); and
the amino acid at the position corresponding to position 242 of SEQ ID NO: 1 is substituted with Trp (Trp242); and
the amino acid at the position corresponding to position 245 of SEQ ID NO: 1 is substituted with Ser (Ser245).

Alternatively or additionally, if the mutant ketoreductase according to the first aspect has more than one amino acid substitution relative to the amino acid sequence of SEQ ID NO: 1, such as three, four, five, six, seven, eight, nine, ten or more amino acid substitutions relative to the amino acid sequence of SEQ ID NO: 1, the amino acid sequence of the mutant ketoreductase of the first aspect of the invention preferably comprises substitutions at positions corresponding to positions 97, 134, 135, 174, 224, 228, 234, 238, 242, 245, 246, 316 and/or 342, particularly positions 97, 134, 224, 238, 242 and/or 245, of SEQ ID NO: 1 in addition to that at position 241.

Accordingly, in a preferred embodiment of the mutant ketoreductase of the first aspect
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val 134), Cys (Cys 134), Ala (Ala 134), Gln (Gln134), or Met (Met134) or unsubstituted,
the amino acid at the position corresponding to position 135 of SEQ ID NO: 1 is substituted with Cys (Cys135) or Thr (Thr135) or unsubstituted,
the amino acid at the position corresponding to position 174 of SEQ ID NO: 1 is substituted with Thr (Thr174), Val (Val 174), Met (Met174), Tyr (Tyr174), Ala (Ala174), Ile (Ile174), Lys (Ly174), Arg (Arg174), Asn (Asn174), Ser (Ser174), or Gln (Gln174), preferably Ala (Ala174), Val (Val174) or Ile (Ile174) or unsubstituted, and/or
the amino acid at the position corresponding to position 224 of SEQ ID NO: 1 is substituted with Ala (Ala224) or unsubstituted,
the amino acid at the position corresponding to position 228 of SEQ ID NO: 1 is substituted with Lys (Lys228), Gln (Gln228), or Arg (Arg228) or unsubstituted,
the amino acid at the position corresponding to position 234 of SEQ ID NO: 1 is substituted with Asp (Asp234), or unsubstituted,
the amino acid at the position corresponding to position 238 of SEQ ID NO: 1 is substituted with Lys (Lys238), Arg (Arg238), Leu (Leu238), Gly (Gly238), His (His238), Asn (Asn238), Trp (Trp238), Asp (Asp238), Thr (Thr238), Ser (Ser238), Gln (Gln238) or Tyr (Tyr238), preferably Lys (Lys238), Arg (Arg238), Leu (Leu238) or Gly (Gly238) more preferably Lys (Lys238) or Arg (Arg238), most preferably Lys (Lys238) or unsubstituted,
the amino acid at the position corresponding to position 241 of SEQ ID NO: 1 is substituted with Met (Met241), Asn (Asn241), Arg (Arg241), Trp (Trp241), Ile(Ile241), Lys (Lys241), His (His241), Gln (Gln241), Gly (241Gly), Asp (Asp241), Ser (Ser 241), Thr (Thr241), Tyr (Tyr241), Cys (Cys241), Ala (Ala241), Val (Val241), or Phe (Phe241), preferably Met (Met241), Gln (Gln241), Cys (Cys241), Tyr (Tyr241), Ser (Ser 241), Thr (Thr241), Val (Val241), or Ala (Ala241), more preferably Met (Met241),
the amino acid at the position corresponding to position 242 of SEQ ID NO: 1 is substituted with Trp (Trp242), Phe (Phe242), Ile (Ile242), Tyr (Tyr242); Cys (Cys242); Val (242Val), Leu (Leu242), Pro (Pro242), Ala (Ala242), Gln (Gln242) or Ser (Ser242) preferably Trp (Trp242), Phe (Phe242), Ile (Ile242), or Tyr (Tyr242), more preferably Trp (Trp242) or Ile (Ile242), most preferably Trp (Trp242),
the amino acid at the position corresponding to position 245 of SEQ ID NO: 1 is substituted with Ser (Ser245), Thr (Thr245), Asn (Asn245), Met (Met245), Asp (Asp245), Trp (Trp245), Phe (Phe245), Cys (Cys245), or His (His245), preferably Ser (Ser245), Thr (Thr245), or Asn (Asn245), more preferably Ser (Ser245) or Thr (Thr245), most preferably Ser (Ser245),
the amino acid at the position corresponding to position 246 of SEQ ID NO: 1 is substituted with Gly (Gly246), Lys (Lys246), preferably Gly (Gly246) or unsubstituted,
the amino acid at the position corresponding to position 316 of SEQ ID NO: 1 is substituted with Met (Met316) or unsubstituted, and/or
the amino acid at the position corresponding to position 342 of SEQ ID NO: 1 is substituted with Met (Met342) or unsubstituted.

In a further preferred embodiment of the mutant ketoreductase of the first aspect
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val134) or unsubstituted,
the amino acid at the position corresponding to position 224 of SEQ ID NO: 1 is substituted with Ala (Ala 224) or unsubstituted,
the amino acid at the position corresponding to position 238 of SEQ ID NO: 1 is substituted with Lys (Lys238) or unsubstituted,
the amino acid at the position corresponding to position 241 of SEQ ID NO: 1 is substituted with Met (Met241),
the amino acid at the position corresponding to position 242 of SEQ ID NO: 1 is substituted with Trp (Trp242), and/or
the amino acid at the position corresponding to position 245 of SEQ ID NO: 1 is substituted with Ser (Ser245).

In a still more preferred embodiment of the mutant ketoreductase of the first aspect
the amino acid at the position corresponding to position 241 of SEQ ID NO: 1 is substituted with Met (Met241),
the amino acid at the position corresponding to position 242 of SEQ ID NO: 1 is substituted with Trp (Trp242), and
the amino acid at the position corresponding to position 245 of SEQ ID NO: 1 is substituted with Ser (Ser245), and optionally wherein
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val134) or unsubstituted,
the amino acid at the position corresponding to position 224 of SEQ ID NO: 1 is substituted with Ala (Ala 224) or unsubstituted,
the amino acid at the position corresponding to position 238 of SEQ ID NO: 1 is substituted with Lys (Lys238) or unsubstituted,
the amino acid at the position corresponding to position 246 of SEQ ID NO: 1 is substituted with Gly (Gly246) or unsubstituted,
the amino acid at the position corresponding to position 316 of SEQ ID NO: 1 is substituted with Met (Met316) or unsubstituted, and/or
the amino acid at the position corresponding to position 342 of SEQ ID NO: 1 is substituted with met (Met342) or unsubstituted.

Especially preferred mutant ketoreductase are defined at least by the following mutations:
the mutant ketoreductase has the mutations Trp97, Met241, Trp242 and Ser245; or
the mutant ketoreductase has the mutations Trp97, Met241, Trp242, Ser245, Met316 and Met342; or
the mutant ketoreductase has the mutations Trp97, Lys238, Met241, Trp242, Ser245, Met316 and Met342; or
the mutant ketoreductase has the mutations Trp97, Lys238, Met241, Trp242, Ser245, Gly246, Met316 and Met342; or
the mutant ketoreductase has the mutations Trp97, 224Ala, Lys238, Met241, Trp242, Ser245, Gly246, Met316 and Met342; or
the mutant ketoreductase has the mutations Trp97, Val134, 224Ala, Lys238, Met241, Trp242, Ser245, Gly246, Met316 and Met342; or
the mutant ketoreductase has the mutations Lys238, Met241, Trp242, Ser245, or
the mutant ketoreductase has the mutations Trp97, Lys238, Met241, Trp242, Ser245.

Further mutations, optionally as defined above or below, may be present.

The mutant ketoreductase according to the first aspect of the invention may further comprise an amino acid sequence that is at least 80%, 85%, 90%, 91%, 92%, 93%, 94%, 95%, 96%, 97%, 98%, or 99%, particularly 100% identical to the amino acid sequence of SEQ ID NO: 2 to 9. Sequence identity and methods for determining sequence identity of the amino acid sequences of two proteins are well-known to the person skilled in the art and also described above.

In a further preferred embodiment of the mutant ketoreductase of the first aspect, the mutant ketoreductase consists of or comprises an amino acid sequence that is at least 85%, 90%, 91%, 92%, 93%, 94%, 95%, 96%, 97%, 98%, or 99%, particularly 100% identical to the amino acid sequence of any of SEQ ID NO: 2 to 9.

In a preferred embodiment of the mutant ketoreductase of the first aspect, the ketoreductase activity relative to the wild-type ketoreductase is increased by at least 1.01, 2.0, 5.0, 10 or even 50-fold. Methods for determining the ketoreductase activity of a protein as well as methods for comparing the ketoreductase activity of two or more proteins are well-known to the person skilled in the art and also described above.

The mutant ketoreductase according to the first aspect of the invention may further have an increased conversion relative to the wild-type ketoreductase at higher substrate loadings such as 2 to 10% [w/w] substrate and at a mutant or wild-type ketoreductase loading of 1-2% [w/w](s/e 5-10) using 2-propanol as cofactor recycling system and 0.1-0.2% [w/w](s/e 50-100) using the glucose/glucose dehydrogenase recycling system.

The term “conversion” as used herein means any substrate to product conversion induced by a ketoreductase, such as by a mutant ketoreductase of the present invention or a wild-type ketoreductase. Such conversion may further depend on various reaction parameters, such as temperature, pressure or the amount of used substrate or ketoreductase enzyme. Suitable conditions and methods are described in the Examples.

Preferably, the conversion of a ketoreductase is determined at 10% [w/w] substrate loading and at a mutant or wild-type ketoreductase loading of 2% [w/w](s/e 5) using 2-propanol as cofactor recycling system. In this context, the abbreviation “s/e” refers to the “substrate-to-enzyme” ratio. A s/e 5 further means that 5 g substrate are used per 1 g enzyme, i.e. substrate and enzyme are used in a ratio of 5/1.

In a further preferred embodiment of the mutant ketoreductase of the first aspect, the mutant ketoreductase has an increased conversion relative to the wild-type ketoreductase at 2 to 10% [w/w] substrate and at a mutant or wild-type ketoreductase loading of 1-2% [w/w](s/e 5-10) using 2-propanol as cofactor recycling system, particularly an increased conversion of at least 1.05, 1.10, 1.20, 1.30, 1.40, 1.50, 1.75, 2.0, 2.5, 3.0, 3.5, 4.0, 4.5, 5.0, 5.5, 6.0, 6.5, 7.0, 7.5, 8.0, 8.5, 9.0, 9.5 or 10-fold compared to the wild-type ketoreductase. An alternative cofactor recycling system may be applied such as glucose and glucose dehydrogenase.

In a preferred embodiment of the mutant ketoreductase of the first aspect, the mutant ketoreductase is capable of the asymmetric reduction of a ketone to the corresponding chiral, non-racemic secondary alcohol, particularly to the pure enantiomers respectively diastereomers of a secondary alcohol.

In a further preferred embodiment the ketone has the formula I

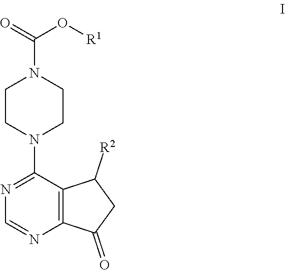

and the chiral alcohol has the formula II

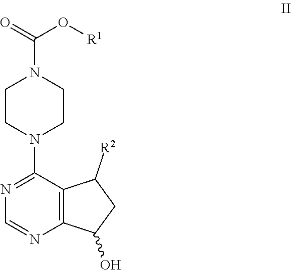

wherein R¹ is C₁₋₄-alkyl and R² is hydrogen or C₁₋₄-alkyl.

The spiral bond
“ [symbol] ”
   stands for “ [symbol] ” or for “ [symbol] ” thus indicating chirality of the molecule.

In a more preferred embodiment R¹ is tert.butyl and R² is methyl.

The mutant ketoreductase can in principle asymmetrically catalyze the formation of both configurations, the S- or R-configuration, of a chiral alcohol, particularly of the chiral alcohol of formula II.

In a preferred embodiment the mutant ketoreductase catalyzes for the formation of R-diastereomers of the chiral alcohol of formula IIa

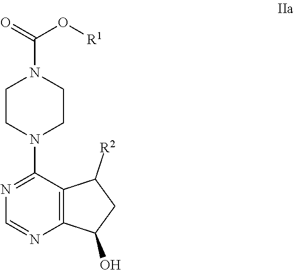

wherein R¹ and R² are as above, more preferably catalyzes for the formation of the chiral alcohol of formula IIb

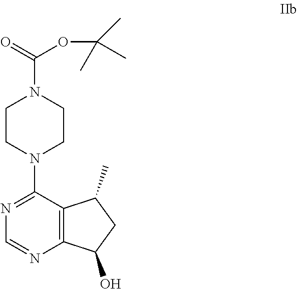 [SMILES: C[C@@H]1C[C@@H](O)c2ncnc(N3CCN(C(=O)OC(C)(C)C)CC3)c21]

An diastereomeric excess of the R,R-diastereomer of the chiral alcohol of at least 95%, 96%, 97%, 98% or 99% can be reached.

Further, the mutant ketoreductase according to the first aspect of the invention may also be combined with a further peptide or protein into a fusion protein. Accordingly, the present invention further concerns a fusion protein comprising the mutant ketoreductase of the present invention.

The fusion protein may further comprise a tag. Tags are attached to proteins for various purposes, e.g. in order to ease purification, to assist in the proper folding in proteins, to prevent precipitation of the protein, to alter chromatographic properties, to modify the protein or to mark or label the protein. The use of a highly pure enzyme as a rule reduces the required enzyme loading. A number of (affinity) tags or (affinity) markers are known at present. Commonly used tags include the Arg-tag, the His-tag, the Strep-tag, the Flag-tag, the T7-tag, the S-tag, the HAT-tag, the GST-tag and the MBP-tag.

In a second aspect the present invention relates to a nucleic acid coding for the mutant ketoreductase according to the first aspect of the invention. Accordingly, the present invention may also relate to a nucleic acid coding for the fusion protein comprising the mutant ketoreductase according to the first aspect of the invention.

The term “nucleic acid” as used herein generally relates to any nucleotide molecule which encodes the mutant ketoreductase of the invention and which may be of variable length. Examples of a nucleic acid of the invention include, but are not limited to, plasmids, vectors, or any kind of DNA and/or RNA fragment(s) which can be isolated by standard molecular biology procedures, including, e.g. ion-exchange chromatography. A nucleic acid of the invention may be used for transfection or transduction of a particular cell or organism.

Nucleic acid molecule of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

Additionally, the nucleic acid may contain one or more modified bases. Such nucleic acids may also contain modifications e.g. in the ribose-phosphate backbone to increase stability and half-life of such molecules in physiological environments. Thus, DNAs or RNAs with backbones modified for stability or for other reasons are “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia.

Furthermore, the nucleic acid molecule encoding the mutant ketoreductase of the invention can be functionally linked, using standard techniques such as standard cloning techniques, to any desired sequence, such as a regulatory sequence, leader sequence, heterologous marker sequence or a heterologous coding sequence to create a fusion protein.

The nucleic acid of the invention may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

The nucleic acid of the invention may be comprised in an expression vector, wherein the nucleic acid is operably linked to a promoter sequence capable of promoting the expression of the nucleic acid in a host cell.

In a preferred embodiment of the nucleic acid codes for a mutant ketoreductase of the first aspect, wherein the mutant ketoreductase consists of or comprises an amino acid sequence that is at least 85%, 90%, 91%, 92%, 93%, 94%, 95%, 96%, 97%, 98%, or 99%, particularly 100% identical to the amino acid sequence of any of SEQ ID NO: 2 to 9.

In a third aspect the present invention relates to a vector comprising a nucleic acid according to the second aspect of the invention. Accordingly, the present invention may also relate to a vector comprising a nucleic acid coding for the fusion protein comprising the mutant ketoreductase according to the first aspect of the invention.

As used herein, the term “vector” generally refers to any kind of nucleic acid molecule that can be used to express a protein of interest in a cell (see also above details on the nucleic acids of the present invention). In particular, the vector of the invention can be any plasmid or vector known to the person skilled in the art which is suitable for expressing a protein in a particular host cell including, but not limited to, mammalian cells, bacterial cell, and yeast cells. A vector of the present invention may also be a nucleic acid which encodes a mutant ketoreductase of the invention, and which is used for subsequent cloning into a respective vector to ensure expression. Plasmids and vectors for protein expression are well known in the art, and can be commercially purchased from diverse suppliers including, e.g., Promega (Madison, WI, USA), Qiagen (Hilden, Germany), Invitrogen (Carlsbad, CA, USA), or MoBiTec (Germany). Methods of protein expression are well known to the person skilled in the art and are, e.g., described in Sambrook et al., 2000, Molecular Cloning: A laboratory manual, Third Edition.

The vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. The vector may be used to transduce, transform or infect a cell, thereby causing the cell to express nucleic acids and/or proteins other than those native to the cell. The vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate vectors, of which numerous types are known in the art, can also be used for this purpose. Methods for obtaining such vectors are well-known (see, e.g. Sambrook et al, supra).

As detailed above, the nucleic acid which encodes a mutant ketoreductase of the invention is operably linked to sequence which is suitable for driving the expression of a protein in a host cell, in order to ensure expression of the protein. However, it is encompassed within the present invention that the claimed vector may represent an intermediate product, which is subsequently cloned into a suitable vector to ensure expression of the protein. The vector of the present invention may further comprise all kind of nucleic acid sequences, including, but not limited to, polyadenylation signals, splice donor and splice acceptor signals, intervening sequences, transcriptional enhancer sequences, translational enhancer sequences, drug resistance gene(s) or alike. Optionally, the drug resistance gene may be operably linked to an internal ribosome entry site (IRES), which might be either cell cycle-specific or cell cycle-independent.

The term “operably linked” as used herein generally means that the gene elements are arranged as such that they function in concert for their intended purposes, e.g. in that transcription is initiated by the promoter and proceeds through the DNA sequence encoding the mutant ketoreductase of the present invention. That is, RNA polymerase transcribes the sequence encoding the mutant ketoreductase into mRNA, which in then spliced and translated into a protein.

The term “promoter sequence” as used in the context of the present invention generally refers to any kind of regulatory DNA sequence operably linked to a downstream coding sequence, wherein said promoter is capable of binding RNA polymerase and initiating transcription of the encoded open reading frame in a cell, thereby driving the expression of said downstream coding sequence. The promoter sequence of the present invention can be any kind of promoter sequence known to the person skilled in the art, including, but not limited to, constitutive promoters, inducible promoters, cell cycle-specific promoters, and cell type-specific promoters.

Moreover, the present invention also comprises a host cell comprising the mutant ketoreductase of the present invention or a fusion protein thereof, the nucleic acid of the second aspect of the invention or the vector of the third aspect of the invention.

A “host cell” of the present invention can be any kind of organism suitable for application in recombinant DNA technology, and includes, but is not limited to, all sorts of bacterial and yeast strain which are suitable for expressing one or more recombinant protein(s). Examples of host cells include, for example, various Bacillus subtilis or E. coli strains. A variety of E. coli bacterial host cells are known to a person skilled in the art and include, but are not limited to, strains such as DH5-alpha, HB101, MV1190, JM109, JM101, or XL-1 blue which can be commercially purchased from diverse suppliers including, e.g., Stratagene (CA, USA), Promega (WI, USA) or Qiagen (Hilden, Germany). A particularly suitable host cell is also described in the Examples, namely E. coli BL21 (DE3) cells. Bacillus subtilis strains which can be used as a host cell include, e.g., 1012 wild type: leuA8 metB5 trpC2 hsdRM1 and 168 Marburg: trpC2 (Trp−), which are, e.g., commercially available from MoBiTec (Germany).

The cultivation of host cells according to the invention is a routine procedure known to the person skilled in the art. That is, a nucleic acid encoding a mutant ketoreductase of the invention can be introduced into a suitable host cell(s) to produce the respective protein by recombinant means. These host cells can by any kind of suitable cells, preferably bacterial cells such as E. coli, which can be cultivated in culture. At a first step, this approach may include the cloning of the respective gene into a suitable vector, such as a vector according to the second aspect of the present invention. Vectors are widely used for gene cloning, and can be easily introduced, i.e. transfected, into bacterial cells which have been made transiently permeable to DNA. After the protein has been expressed in the respective host cell, the cells can be harvested and serve as the starting material for the preparation of a cell extract containing the protein of interest. A cell extract containing the protein of interest is obtained by lysis of the cells. Methods of preparing a cell extract by means of either chemical or mechanical cell lysis are well known to the person skilled in the art, and include, but are not limited to, e.g. hypotonic salt treatment, homogenization, or ultrasonication.

In a fourth aspect the present invention relates to a method for the enzymatic reduction of a ketone and the formation of a chiral alcohol in the presence of mutant ketoreductase of the present invention.

In a further preferred embodiment the ketone of the formula I

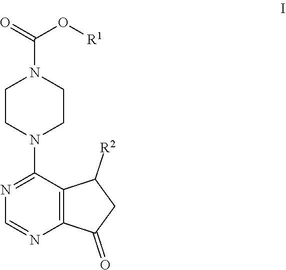

is reduced and the chiral alcohol of the formula II

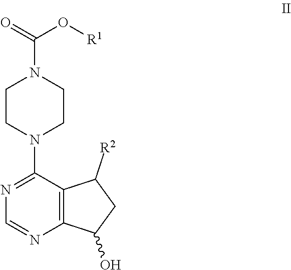

or, more preferably the chiral alcohol of the formula IIa

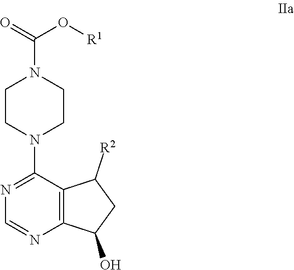

wherein R¹ is C₁₋₄-alkyl and R² is hydrogen or C₁₋₄-alkyl is formed.

Even more preferably the chiral alcohol of formula IIb

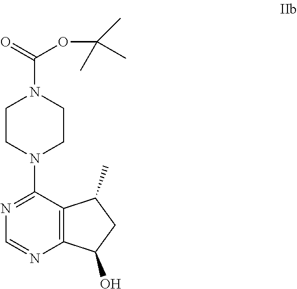 [SMILES: C[C@@H]1C[C@@H](O)c2ncnc(N3CCN(C(=O)OC(C)(C)C)CC3)c21]

is formed by asymmetrically reducing the ketone of formula Ib

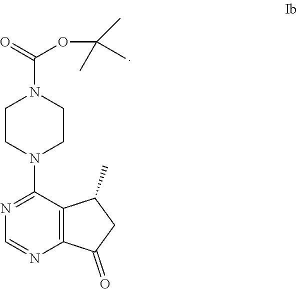 [SMILES: C[C@@H]1CC(=O)c2ncnc(N3CCN(C(=O)OC(C)(C)C)CC3)c21]

Preferably, the resulting chiral alcohol of formula IIb is the (R, R)-diastereomer.

The enzymatic reduction with the mutant ketoreductase usually take place in the presence of NADH or NADPH as cofactor. More preferably, NADP⁺ is used and its reduced form NADPH is regenerated in-situ.

The oxidized cofactor is as a rule continuously regenerated with a secondary alcohol as final reductant, so-called cosubstrate or in-situ cofactor recycling systems, i.e. glucose dehydrogenase and glucose as final reductant, as commonly known by one of ordinary skill in the art in the field of the invention.

Typical co-substrates can be selected from 2-propanol, 2-butanol, pentan-1,4-diol, 2-pentanol, 4-methyl-2-pentanol, 2-heptanol, hexan-1,5-diol, 2-heptanol or 2-octanol, preferably 2-propanol. The acetone formed when 2-propanol is used as co-substrate which can in a further preferred embodiment be continuously removed from the reaction mixture.

The cofactor loading i.e. the ratio substrate (ketone) to cofactor (s/c) can vary between 10 and 250, preferably between 50 and 200, most preferably is 100.

In a particular embodiment of the present invention, the enzymatic reduction is performed in an aqueous buffer medium in the presence of the co-substrate i.e. preferably in the presence of 2-propanol. The concentration of the co-substrate is typically in the range of 5% [v/v] to 20% [v/v], preferably 8% [v/v].

In a particular embodiment of the present invention, the enzymatic reduction is performed in an aqueous buffer medium in the presence of glucose and glucose dehydrogenase. The concentration of glucose is typically in the range 0.2 M to 2 M, respectively at least 1.1 equivalents with respect to the target ketone. In order to neutralize the formed gluconic acid, the addition of base is necessary to constantly adjust to the target pH.

Suitable buffers can be selected from acidic to neutral buffers such as 2-morpholin-4-ethanesulfonic acid, ammonium acetate, acetate, phosphate, 1,4-Piperazinediethanesulfonic acid, which allow to keep the pH of the reaction in the range between pH 6 and pH 10, particularly between 6.8 to 7.2, more particularly about 7.0 to 7.2.

The substrate loading, i.e. the loading of the ketone may be selected between 1% and 20% [w/w], preferably 10% [w/w] and the ratio substrate to enzyme (s/e) is dependent on the cofactor recycling system applied, respectively the final reductant.

In case of 2-propanol as final reductant, the ratio substrate to enzyme (s/e) can be selected between 4 and 50, preferable between 4 and 10. In case of glucose as final reductant, the ratio substrate to enzyme (s/e) can be selected between 10 and 200, preferable between 50 and 100.

The reaction temperature is usually kept in a range between 10° C. and 50° C., preferably between 20° C. and 35° C., more preferably between 23° C. and 30° C.

Upon termination of the reaction the resulting chiral alcohol can be conventionally worked up by extraction or preferred by filtration.

The synthesis of ipatasertib from the chiral alcohol formed in the enzymatic synthesis according to the present invention can follow the synthesis scheme 3, page 42 of the WO2008006040A1 and the corresponding examples applying average skill in the art.

In a fifth aspect the present invention relates to the use of the methods of the present invention (enzymatic reduction of a ketone and of the formation of a chiral alcohol in the presence of mutant ketoreductase) for the preparation of serine/threonine protein kinase inhibitors of the formula

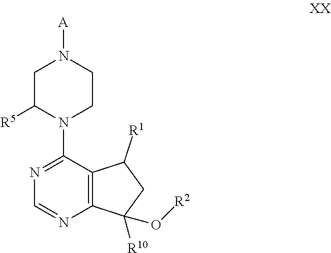

as illustrated e.g. in the PCT International Application WO 2008/006040 A1.

Particularly A, R¹, R², R⁵ and R¹⁰ may be as defined in claim 1 of the PCT International Application WO 2008/006040 A1:
R¹ may be H, methyl, ethyl, vinyl, CF₃, CHF₂ or CH₂F;
R² may be H or methyl;
R⁵ may be H, methyl, ethyl, or CF₃;
R¹⁰ may be H or methyl; and
A may be

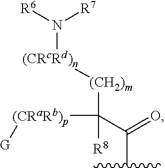

wherein G is phenyl optionally substituted by one to four R⁹ groups or a 5-6 membered heteroaryl optionally substituted by a halogen; R⁶ and R⁷ are independently H, OCH₃, (C₃-C₆ cycloalkyl)-(CH₂), (C₃-C₆ cycloalkyl)-(CH₂CH₂), V—(CH₂)₀₋₁ wherein V is a 5-6 membered heteroaryl, W—(CH₂)₁₋₂ wherein W is phenyl optionally substituted with F, Cl, Br, I, O-methyl, CF₃ or methyl, C₃-C₆-cycloalkyl optionally substituted with C₁-C₃ alkyl or O(C₁-C₃ alkyl), hydroxy-(C₃-C₆-cycloalkyl), fluoro-(C₃-C₆-cycloalkyl), CH(CH₃)CH(OH)phenyl, 4-6 membered heterocycle optionally substituted with F, OH, C₁-C₃ alkyl, cyclopropylmethyl or C(═O)(C₁-C₃ alkyl), or C₁-C₆-alkyl optionally substituted with one or more groups independently selected from OH, oxo, O(C₁-C₆-alkyl), CN, F, NH₂, NH(C₁-C₆-alkyl), N(C₁-C₆-alkyl)₂, cyclopropyl, phenyl, imidazolyl, piperidinyl, pyrrolidinyl, morpholinyl, tetrahydrofuranyl, oxetanyl or tetrahydropyranyl, or R⁶ and R⁷ together with the nitrogen to which they are attached form a 4-7 membered heterocyclic ring optionally substituted with one or more groups independently selected from OH, halogen, oxo, CF₃, CH₂CF₃, CH₂CH₂OH, O(C₁-C₃ alkyl), C(═O)CH₃, NH₂, NHMe, N(Me)₂, S(O)₂CH₃, cyclopropylmethyl and C₁-C₃ alkyl; R^{a} and R^{b} are H, or R^{a} is H, and R^{b} and R⁶ together with the atoms to which they are attached form a 5-6 membered heterocyclic ring having one or two ring nitrogen atoms; R^{c} and R^{d} are H or Me, or R^{c} and R^{d} together with the atom to which they are attached from a cyclopropyl ring; R³ is H, Me, F or OH, or R³ and R⁶ together with the atoms to which they are attached form a 5-6 membered heterocyclic ring having one or two ring nitrogen atoms; each R⁹ is independently halogen, C₁-C₆-alkyl, C₃-C₆-cycloalkyl, O—(C₁-C₆-alkyl), CF₃, OCF₃, S(C₁-C₆-alkyl), CN, OCH₂-phenyl, CH₂O-phenyl, NH₂, NH—(C₁-C₆-alkyl), N—(C₁-C₆-alkyl)₂, piperidine, pyrrolidine, CH₂F, CHF₂, OCH₂F, OCHF₂, OH, SO₂(C₁-C₆-alkyl), C(O)NH₂, C(O)NH(C₁-C₆-alkyl), and C(O)N(C₁-C₆-alkyl)₂ and m, n and p are independently 0 or 1.

The enzymatic reduction is particularly promising for clinical AKT inhibitor candidate ipatasertib (CAS Reg. No. 1001264-89-6), which has the formula X.

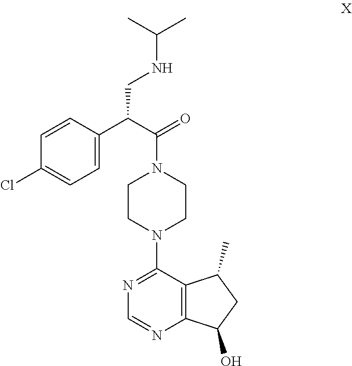 [SMILES: CC(C)NC[C@H](C(=O)N1CCN(c2ncnc3c2[C@H](C)C[C@H]3O)CC1)c1ccc(Cl)cc1]

With respect to the use of the present invention it is referred to the terms, examples and specific embodiments used in the context of the other aspects of the present disclosure, which are also applicable to this aspect. Particularly, the mutant ketoreductase according to the present invention or the fusion protein thereof may be used as detailed with respect to the methods of the present invention.

Unless defined otherwise, all technical and scientific terms and any acronyms used herein have the same meanings as commonly understood by one of ordinary skill in the art in the field of the invention. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The invention is not limited to the particular methodology, protocols, and reagents described herein because they may vary. Although any methods and materials similar or equivalent to those described herein can be used in the practice of the present invention, the preferred methods, and materials are described herein. Further, the terminology used herein is for the purpose of describing particular embodiments only and is not intended to limit the scope of the present invention.

As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively. Similarly, the word “or” is intended to include “and” unless the context clearly indicates otherwise. The term “plurality” refers to two or more.

The following Figures and Examples are intended to illustrate various embodiments of the invention. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLES

### Example 1: Enzyme Production

Deep-well plate cultivation: E. coli cells expressing wild-type and mutant ketoreductases were cultivated in a 96-deep well plate format for screening purposes. Precultures were started by inoculation of fresh single transformants or glycerol stocks into 500 μL Luria-Bertani (LB) medium containing 100-200 mg/L ampicillin, followed by incubation at 28° C. with shaking at 300 rpm for 18 h (Duetz system, Kuehner shaker, 5 cm shaking diameter). Main cultures were started by inoculation of 8-25 μL preculture into 500 PL ZYM-5052 autoinduction medium without trace elements (10 g/L peptone, 5 g/L yeast extract, 5 g/L glycerol, 0.55 g/L glucose monohydrate, 2.1 g/L lactose monohydrate, 10.6 g/L sodium phosphate dibasic salt, 3.4 g/L potassium phosphate monobasic salt, 2.15 g/L ammonium chloride, 0.59 g/L sodium chloride, 0.663 g/L ammonium sulfate, 2 mM magnesium sulfate) supplemented with 100-200 mg/L ampicillin. The cultures were incubated at 20° C., 300 rpm for 20 h in the same shaker. Prior to cell harvesting, optical cell densities at 600 nm were measured.

Cells were disrupted by addition of 200 μL lysis buffer (0.1 M potassium phosphate buffer pH 7, 2 mM MgCl₂, 1 mg/mL lysozyme from chicken egg white, 0.75 mg/mL polymyxin B sulfate and 0.2 mg/mL DNase I). Cell suspensions were incubated at 30° C. with shaking at 300 rpm for 1 h (Duetz system, Kuehner shaker), followed by centrifugation at 4° C., 3,220 g for 30 min. Supernatants of deep-well plate-cultivated cells were immediately used for the UV-based activity assay or in 0.2 mL-scale biocatalytic reactions at 10% [w/w] substrate loading.

Shake flask cultivation: E. coli cells expressing wild-type and mutant ketoreductases were cultivated in shake flasks for ≥1 mL-scale biocatalytic reactions. One E. coli transformant served to inoculate 20 mL LB containing 100 mg/L ampicillin, followed by incubation at 37° C., 180 rpm overnight. Precultures (5 mL) were used to inoculate 2 L Erlenmeyer flasks containing 500 mL Terrific Broth (TB) medium with 100 mg/L ampicillin, followed by incubation at 37° C., 180 rpm until an OD 600 nm of 0.6-0.8 was reached. After addition of 1 mM isopropyl β-D-1-thiogalactopyranoside (IPTG), the cultures were incubated at 25 or 30° C. for 24 h. Cells were harvested by centrifugation at 4° C., 9,300 g for 45 min. Pellets were weighed and resuspended in 2 mM potassium phosphate buffer pH 7 containing 0.04 mM MgCl₂ in a 1:2 biomass-buffer ratio. Cells were sonicated and centrifuged at 4° C., 30,000 g for 20 min. Supernatants were collected for lyophilization.

Lyophilization of cell lysates was performed overnight using an Alpha 2-4 LDplus (Christ) set under −85° C. and 0.14 mbar. Lyophilized lysates were immediately used for biocatalytic reactions or stored at −20° C.

### Example 2: Determination of Enzyme Activity and Selectivity

### UV Screening in Small Scale Reaction

Reductase activity was measured in 96-well Greiner microtiter plates using a spectrophotometer. Reactions were performed in a total volume of 200 μL containing: (1) 178 μL of 0.1 M potassium phosphate buffer pH 7 with 2 mM MgCl₂ and 0.01 mg/mL NADP⁺ (as sodium salt); (2) 6 μL of clarified lysate (pure or diluted in 0.1 M potassium phosphate buffer pH 7 containing 2 mM MgCl₂, for a final lysate concentration of 3%, 1.5%, 1%, 0.5% or 0.25% [v/v][v/v]); and (3) 16 μL of a stock solution containing 1.25 mg/mL of the ketone of formula Ib in 2-propanol [v/v].

The assay was run with orbital shaking at 432 rpm and at temperatures oscillating between 28° C. and 32° C. (room temperature plus 5° C. caused by shaking). Depletion of the ketone of formula Ib was followed at 340 nm and recorded every 5 min for 80 min. Slopes [ΔA/min]) within the linear range were used for fold-increase over the parent (FIOP) calculations. The parent can be wild-type (FIOWT) or a different variant. The following tables show FIOWT or FIOP values for various single and multiple mutants in comparison to the wild-type. Diastereomeric excess (de) values for the chiral alcohol of formula IIb (R,R-trans alcohol) of the hits were verified by HPLC-UV analysis of the UV screening assay samples (see section: HPLC analysis of substrate and products). In case of all mutants displayed in Tables 1-20, the de of chiral alcohol of formula IIb was ≥99.5%.

TABLE 1
Variants with single mutation at position
241 (Single site mutagenesis)

| AA | FIOWT |
|---|---|
| F - Phe | 1.00 |
| I - Ile | 1.16 |
| K - Lys | 1.20 |
| L - Leu (wild-type) | 1.00 |
| M - Met | 1.22 |
| N - Asn | 1.14 |
| R - Arg | 1.20 |
| W - Trp | 1.20 |

UV screening assays were carried out with 3% [v/v] lysate.

TABLE 2
Variants with mutation at position 241 and at least
one further mutation (Combinatorial site mutagenesis)

| Phe 97 | Leu 174 | Ala 238 | Leu 241 | Met 242 | Glu 245 | FIOWT |
|---|---|---|---|---|---|---|
| W—Trp | L—Leu | A—Ala | M—Met | W—Trp | S—Ser | 5.0 |
| W—Trp | L—Leu | A—Ala | A—Ala | W—Trp | S—Ser | 4.9 |
| W—Trp | L—Leu | A—Ala | C—Cys | W—Trp | S—Ser | 4.8 |
| W—Trp | L—Leu | A—Ala | Y—Tyr | W—Trp | S—Ser | 4.3 |
| W—Trp | L—Leu | A—Ala | V—Val | Y—Tyr | T—Thr | 3.4 |
| W—Trp | L—Leu | A—Ala | Q—Gln | M—Met | N—Asn | 2.9 |
| W—Trp | L—Leu | A—Ala | W—Trp | V—Val | W—Trp | 2.7 |
| W—Trp | L—Leu | A—Ala | F—Phe | Y—Tyr | T—Thr | 2.6 |
| W—Trp | L—Leu | A—Ala | H—His | F—Phe | S—Ser | 2.4 |
| W—Trp | L—Leu | R—Arg | G—Gly | M—Met | W—Trp | 3.1 |
| W—Trp | W—Trp | K—Lys | D—Asp | I—Ile | F—Phe | 2.9 |
| W—Trp | I—Ile | W—Trp | S—Ser | P—Pro | N—Asn | 2.7 |
| W—Trp | L—Leu | L—Leu | T—Thr | F—Phe | C—Cys | 2.6 |

UV screening assays were carried out with 3% [v/v] lysate.

TABLE 3
Variants with mutation at position 241 and mutations on positions
97, 242 and 245 (Combinatorial site mutagenesis)

| Phe 97 | Leu 241 | Met 242 | Glu 245 | FIOWT |
|---|---|---|---|---|
| W - Trp | M - Met | W - Trp | S - Ser | 8.3 |
| W - Trp | Q - Gln | W - Trp | S - Ser | 8.0 |
| W - Trp | S - Ser | W - Trp | S - Ser | 7.4 |
| W - Trp | A - Ala | W - Trp | S - Ser | 7.2 |
| W - Trp | T - Thr | W - Trp | S - Ser | 6.1 |
| W - Trp | C - Cys | W - Trp | S - Ser | 5.8 |
| W - Trp | V - Val | W - Trp | S - Ser | 5.4 |
| W - Trp | Y - Tyr | W - Trp | S - Ser | 5.1 |
| W - Trp | L - Leu | W - Trp | T - Thr | 4.7 |
| W - Trp | V - Val | Y - Tyr | T - Thr | 4.2 |
| W - Trp | C - Cys | W - Trp | T - Thr | 4.1 |
| W - Trp | Q - Gln | W - Trp | T - Thr | 3.7 |
| W - Trp | T - Thr | F - Phe | T - Thr | 3.3 |
| W - Trp | A - Ala | W - Trp | T - Thr | 3.2 |

UV screening assays were carried out with 1.5% [v/v] lysate.

TABLE 4
Variants with single mutation at position
242 (Single site mutagenesis)

| AA | FIOWT |
|---|---|
| F - Phe | 2.63 |
| I - Ile | 1.22 |
| M - Met (wild-type) | 1.00 |
| W - Trp | 1.66 |

UV screening assays were carried out with 3% [v/v] lysate.

TABLE 5
Variants with mutation at position 242 and at least
one further mutation (Combinatorial site mutagenesis)

| Phe 97 | Leu 174 | Ala 238 | Leu 241 | Met 242 | Gln 245 | FIOWT |
|---|---|---|---|---|---|---|
| W—Trp | L—Leu | A—Ala | L—Leu | P—Pro | T—Thr | 2.1 |
| W—Trp | L—Leu | A—Ala | L—Leu | A—Ala | T—Thr | 1.9 |
| W—Trp | L—Leu | A—Ala | V—Val | Y—Tyr | T—Thr | 3.4 |
| W—Trp | L—Leu | A—Ala | W—Trp | V—Val | W—Trp | 2.7 |
| W—Trp | L—Leu | N—Asn | R—Arg | L—Leu | Q—Gln | 2.7 |
| W—Trp | L—Leu | L—Leu | T—Thr | F—Phe | C—Cys | 2.6 |
| W—Trp | L—Leu | N—Asn | R—Arg | L—Leu | Q—Gln | 2.7 |
| W—Trp | K—Lys | G—Gly | T—Thr | C—Cys | T—Thr | 1.6 |
| W—Trp | L—Leu | A—Ala | L—Leu | C—Cys | W—Trp | 1.5 |
| W—Trp | C—Cys | L—Leu | N—Asn | Q—Gln | N—Asn | 1.4 |

UV screening assays were carried out with 3% [v/v] lysate.

TABLE 6
Variants with single mutation at position
245 (Single site mutagenesis)

| AA | FIOWT |
|---|---|
| C - Cys | 1.12 |
| H - His | 1.38 |
| N - Asn | 2.35 |
| Q - Gln (wild-type) | 1.00 |
| S - Ser | 1.35 |
| T - Thr | 3.55 |

UV screening assays were carried out with 3% [v/v] lysate.

TABLE 7
Variants with mutation at position 245 and at least
one further mutation (Combinatorial site mutagenesis)

| Phe 97 | Leu 174 | Ala 238 | Leu 241 | Met 242 | Gln 245 | FIOWT |
|---|---|---|---|---|---|---|
| W—Trp | I—Ile | K—Lys | H—His | W—Trp | D—Asp | 3.4 |
| W—Trp | L—Leu | R—Arg | G—Gly | M—Met | W—Trp | 3.1 |
| W—Trp | W—Trp | K—Lys | D—Asp | I—Ile | F—Phe | 2.9 |
| W—Trp | L—Leu | L—Leu | T—Thr | F—Phe | C—Cys | 2.6 |
| W—Trp | Q—Gln | D—Asp | R—Arg | I—Ile | M—Met | 2.0 |

UV screening assays were carried out with 3% [v/v] lysate.

TABLE 8
Variants with single mutation at position
97 (Single site mutagenesis)

| AA | FIOWT |
|---|---|
| F- Phe (wild-type) | 1.00 |
| W - Trp | 1.41 |

UV screening assays were carried out with 3% [v/v] lysate.

TABLE 9a
Variants with single mutation at position 97 and at least
one further mutation (Combinatorial site mutagenesis)

| | | | | | FIO (vs. | |
|---|---|---|---|---|---|---|
| Phe 97 | Ala 238 | Leu 241 | Met 242 | Gln 245 | E00158) | FIOWT^{[calculated]} |
| F—Phe | K—Lys | M—Met | W—Trp | S—Ser | 0.78 | 17.3 |
| W—Trp | K—Lys | M—Met | W—Trp | S—Ser | 0.97 | 21.5 |

E00158 contains the following mutations:
W97_K238_M241_W242_S245_M316_M342
UV screening assays were carried out with 0.25% [v/v] lysate.

TABLE 9b
Variants with single mutation at position 97, His-tag and at least
three further mutations (Combinatorial site mutagenesis)

| | 97 | 134 | 224 | 238 | 241 | 242 | 245 | 246 | 316 | 342 | FIOP | FIOWT^{a} |
|---|---|---|---|---|---|---|---|---|---|---|---|---|
| WT | F | T | S | A | L | M | Q | Y | L | T | 1.0 | 1 |
| E00128 | W | T | S | A | M | W | S | Y | L | T | 8.0 | 8 |
| E00144 | W | T | S | A | M | W | S | Y | M | M | 1.1 | 9 |
| E00158 | W | T | S | K | M | W | S | Y | M | M | 2.5 | 22 |
| E00174 | W | T | S | K | M | W | S | G | M | M | 1.1 | 24 |
| E00184 | W | T | A | K | M | W | S | G | M | M | 1.2 | 29 |
| E00184 | W | V | A | K | M | W | S | G | M | M | 2.0 | 58 |

^{a}Values estimated from FIOP

TABLE 9c
Kinetic characterization of variants with single
mutation at position 97, His-tag and at least one
three mutations (Combinatorial site mutagenesis)

| | app. K_{M} | app. k_{cat} | app. k_{cat}/K_{M} | |
|---|---|---|---|---|
| variant | [μM] | [min−¹] | [min⁻¹ mM⁻¹] | rel. k_{cat} |
| WT | 11.9 ± 1.1 | 0.49 ± 0.02 | 41 | 1 |
| E00128 | 162.8 ± 7.8 | 5.8 ± 0.1 | 35.5 | 12 |
| E00144 | 175.2 ± 9.9 | 6.2 ± 0.2 | 35.6 | 13 |
| E00158 | 299.7 ± 7.9 | 15.3 ± 0.4 | 51.2 | 31 |
| E00174^{[a]} | 1668 ± 103 | 45.8 ± 2.4 | 27.5 | 94 |
| E00184 | 471.7 ± 34.6 | 21.3 ± 1.0 | 45.2 | 43 |
| E00184 | 143.5 ± 5.5 | 31.5 ± 0.8 | 219.7 | 64 |

^{[a]}substrate saturation as not reached

TABLE 10
Variants with single mutation at position 134 in
mutant E00184 (Single site mutagenesis in E00184)

| Thr 134 | FIOP (vs. E00184) | FIOWT^{[calculated]} |
|---|---|---|
| T - Thr | 1.0 | 29.0 |
| V - Val | 2.0 | 58.0 |
| C - Cys | 1.3 | 37.7 |
| A - Ala | 1.2 | 34.8 |
| Q - Gln | 1.1 | 31.9 |
| M - Met | 1.1 | 31.9 |

E00184 contains the following mutations:
W97_A224_K238_M241_W242_S245_G246_M316_M342
UV screening assays were carried out with 0.25% [v/v] lysate.

TABLE 11
Variants with mutation at positions 134 and 135
in mutant E00184 (Two-site mutagenesis in E00184)

| Thr 134 | Val 135 | FIOP (vs. E00184) | FIOWT^{[calculated]} |
|---|---|---|---|
| V—Val | V—Val | 2.0 | 58.0 |
| V—Val | C—Cys | 1.5 | 43.5 |
| V—Val | T—Thr | 1.5 | 43.5 |

E00184 contains the following mutations:
W97_A224_K238_M241_W242_S245_G246_M316_M342
UV screening assays were carried out with 0.25% [v/v] lysate.

TABLE 12
Variants with single mutation at position
174 (Single site mutagenesis)

| AA | FIOWT |
|---|---|
| A—Ala | 1.29 |
| I—Ile | 1.04 |
| L—Leu (wild-type) | 1.00 |
| T—Thr | 1.01 |
| V—Val | 1.09 |
| Y—Tyr | 1.19 |

UV screening assays were carried out with 3% [v/v] lysate.

TABLE 13
Variants with mutation at position 174 and at least
one further mutation (Combinatorial site mutagenesis)

| Phe 97 | Leu 174 | Ala 238 | Leu 241 | Met 242 | Gln 245 | FIOWT |
|---|---|---|---|---|---|---|
| W—Trp | N—Asn | A—Ala | L—Leu | M—Met | T—Thr | 2.7 |
| W—Trp | S—Ser | A—Ala | L—Leu | M—Met | N—Asn | 2.3 |
| W—Trp | Q—Gln | A—Ala | L—Leu | A—Ala | T—Thr | 1.9 |
| W—Trp | T—Thr | Y—Tyr | W—Trp | M—Met | Q—Gln | 2.8 |
| W—Trp | V—Val | Y—Tyr | L—Leu | M—Met | Q—Gln | 2.8 |
| W—Trp | M—Met | L—Leu | R—Arg | M—Met | Q—Gln | 2.4 |
| W—Trp | Y—Tyr | K—Lys | N—Asn | M—Met | Q—Gln | 2.2 |
| W—Trp | A—Ala | L—Leu | M—Met | M—Met | Q—Gln | 2.1 |
| W—Trp | I—Ile | N—Asn | L—Leu | M—Met | Q—Gln | 2.1 |
| W—Trp | T—Thr | R—Arg | M—Met | M—Met | Q—Gln | 2.0 |
| W—Trp | K—Lys | Y—Tyr | Q—Gln | M—Met | Q—Gln | 2.0 |
| W—Trp | R—Arg | H—His | R—Arg | M—Met | Q—Gln | 1.8 |

UV screening assays were carried out with 3% [v/v] lysate.

TABLE 14
Variants with single mutation at position 224 in mutant
E00174 (Single site mutagenesis in mutant E00174)

| Ser 224 | FIOP (vs. E00174) | FIOWT^{[calculated]} |
|---|---|---|
| S—Ser | 1.0 | 24.4 |
| A—Ala | 1.2 | 29.0 |

E00174 contains the following mutations:
W97_K238_M241_W242_S245_G246_M316_M342
UV screening assays were carried out with 0.25% [v/v] lysate.

TABLE 15
Variants with single mutation at position 228 in mutant
E00158 (Single site mutagenesis in mutant E00158)

| Met 228 | FIOP (vs. E00158) | FIOWT^{[calculated]} |
|---|---|---|
| M—Met | 1.00 | 22.2 |
| K—Lys | 1.08 | 24.0 |
| Q—Gln | 1.14 | 25.3 |
| R—Arg | 1.37 | 30.4 |

E00158 contains the following mutations:
W97_K238_M241_W242_S245_M316_M342
UV screening assays were carried out with 0.25% [v/v] lysate.

TABLE 16
Variants with single mutation at position 234 in mutant
E00158 (Single site mutagenesis in mutant E00158)

| Glu 234 | FIOP (vs. E00158) | FIOWT^{[calculated]} |
|---|---|---|
| E—Glu | 1.0 | 22.2 |
| D—Asp | 1.1 | 24.0 |

E00158 contains the following mutations:
W97_K238_M241_W242_S245_M316_M342
UV screening assays were carried out with 0.25% [v/v] lysate.

TABLE 17
Variants at position 238 in mutant E00144
(Single site mutagenesis in mutant E00144)

| Ala 238 | FIOP (vs. E00144) | FIOWT^{[calculated]} |
|---|---|---|
| A—Ala | 1.0 | 9.0 |
| K—Lys | 2.5 | 22.2 |
| G—Gly | 1.9 | 17.3 |
| R—Arg | 1.7 | 15.3 |

E00144 contains the following mutations:
W97_M241_W242_S245_M316_M342
UV screening assays were carried out with 0.25% [v/v] lysate.

TABLE 18
Variants with mutation at position 238 and at least
one further mutation (Combinatorial site mutagenesis)

| Phe 97 | Leu 174 | Ala 238 | Leu 241 | Met 242 | Gln 245 | FIOWT |
|---|---|---|---|---|---|---|
| W—Trp | L—Leu | K—Lys | N—Asn | I—Ile | Q—Gln | 3.0 |
| W—Trp | L—Leu | N—Asn | R—Arg | L—Leu | Q—Gln | 2.7 |
| W—Trp | L—Leu | L—Leu | L—Leu | M—Met | Q—Gln | 2.6 |
| W—Trp | L—Leu | H—His | H—His | I—Ile | Q—Gln | 2.4 |
| W—Trp | L—Leu | N—Asn | K—Lys | I—Ile | Q—Gln | 1.7 |
| W—Trp | V—Val | Y—Tyr | L—Leu | M—Met | Q—Gln | 2.8 |
| W—Trp | V—Val | Q—Gln | H—His | M—Met | Q—Gln | 2.3 |
| W—Trp | T—Thr | R—Arg | M—Met | M—Met | Q—Gln | 2.0 |
| W—Trp | V—Val | T—Thr | L—Leu | M—Met | Q—Gln | 1.8 |
| W—Trp | V—Val | S—Ser | L—Leu | M—Met | Q—Gln | 1.8 |
| W—Trp | L—Leu | L—Leu | M—Met | M—Met | Q—Gln | 1.8 |
| W—Trp | I—Ile | G—Gly | C—Cys | Y—Tyr | T—Thr | 2.4 |
| W—Trp | M—Met | D—Asp | A—Ala | M—Met | T—Thr | 2.3 |
| W—Trp | I—Ile | W—Trp | S—Ser | P—Pro | N—Asn | 1.8 |

UV screening assays were carried out with 3% [v/v] lysate.

TABLE 19
Variants at position 246 in mutant E00158
(Single site mutagenesis in mutant E00158)

| Tyr 246 | FIOP (vs. E00158) | FIOWT^{[calculated]} |
|---|---|---|
| Y—Tyr | 1.0 | 22.2 |
| G—Gly | 1.1 | 24.4 |
| K—Lys | 1.1 | 24 |

E00158 contains the following mutations:
W97_K238_M241_W242_S245_M316_M342
UV screening assays were carried out with 0.25% [v/v] lysate.

TABLE 20
Variants with mutations at positions 316 and 342 and at least
one further mutation (Combinatorial site mutagenesis)

| | | | | FIOWT | FIOWT |
|---|---|---|---|---|---|
| | | | | no surface | with surface |
| | | | | residue mutations | residue mutations |
| Phe 97 | Leu 241 | Met 242 | Gln 245 | (Leu316_Thr342) | (Met316_Met342) |
| F—Phe | L—Leu | M—Met | Q—Gln | 1.0 | 1.4 |
| F—Phe | L—Leu | M—Met | T—Thr | 3.8 | 5.1 |
| W—Trp | M—Met | W—Trp | S—Ser | 8.0 | 8.5 |
| W—Trp | C—Cys | W—Trp | S—Ser | 5.8 | 6.6 |
| W—Trp | Y—Tyr | W—Trp | S—Ser | 5.1 | 5.6 |
| W—Trp | V—Val | Y—Tyr | T—Thr | 4.2 | 4.9 |

UV screening assays were carried out with 1.5% [v/v] lysate.

### HPLC Analysis of Substrate and Products

Achiral method for the determination of diastereomeric excess (de): Reaction mixtures were quenched with HPLC-grade methanol in a convenient ratio according to the substrate concentration. After protein precipitation, samples were centrifuged at 4° C., 3,300 g for 10 min. Supernatants were analyzed by HPLC-UV at 260 nm on an Agilent 1290 HPLC system using one of the following methods:
i. Kinetex XB-C₁₈ column (50 mm×4.6 mm, 2.6 μm), using water and methanol as solvents A and B, respectively. The column was heated at 50° C., the flow rate was 0.8 mL/min, and the injection volume was 2 μL. The following gradient was applied: 0-0.1 min, B=50%; 0.1-4 min, B=50-58%; 4-4.1 min, B=58-95%; 4.1-4.6 min, B=95%; 4.6-4.7 min, B=95-50%; 4.7-5 min, B=50%, 5-5.1 min, B=50-95%; 5.1-5.4 min, B=95%; 5.4-5.5 min, B=95-50%; and 5.5-5.9 min, B=50%.
ii. Agilent InfinityLab Poroshell 120 Eclipse EC-C₁₈ column (50 mm×3.0 mm×2.7 μm), using water and methanol as solvents A and B, respectively. The column was heated at 50° C., the flow rate was 0.8 mL/min, and the injection volume was 2 μL. The following gradient was applied: 0-0.1 min, B=40%; 0.1-6 min, B=40-52.7%; 6-6.2 min, B=52.7-95%; 6.2-7.2 min, B=95%; 7.2-8 min, B=95-40%: 8-9 min, B=40%.
iii. Kinetex EVO-C₁₈ column (50 mm×4.6 mm, 5 μm), using water and methanol as solvents A and B, respectively. The column was heated at 40° C., the flow rate was 2 mL/min, and the injection volume was 1 μL. The following isocratic method was applied: 0-7.5 min, B=40%.

The method i or ii was used after UV assay screening or 0.2 mL-scale biocatalytic reactions. Standards corresponding to of the ketone of formula Ib, the chiral alcohol of formula IIb and the (R,S)-cis-alcohol product derived from of the ketone of formula Ib were treated as the samples prior to the HPLC-UV analysis. For quick verification of selectivity from UV assay samples, diastereomeric excess (de) values were estimated from the relative peak areas of the alcohol products (as area %, abbreviated as a %). In case of 0.2 mL-scale biocatalytic reactions, conversions were calculated using a calibration curve of the chiral alcohol of formula IIb. Method iii was applied for ≥1 mL-scale reactions. Conversion and diastereomeric excess values were determined from the relative peak areas.

TABLE 21
Reductase performance and diastereoselectivity of ketoreductases
from different fungal species towards the ketone of formula
Ib in comparison with SEQ ID NO: 1 (UniProt ID: Q9UUN9)

| | NCBI/ | FIO | de [%] |
|---|---|---|---|
| Fungal species | UniProt ID | SEQ ID NO: 1 | (IIb) |
| Scheffersomyces stipitis | ABN64930 | 2.60 | 98.0 |
| Clavispora lusitaniae | EEQ37269 | 2.10 | 93.5 |
| Meyerozyma guilliermondii | EDK39148 | 1.60 | 42.8 |
| Sporidiobolus salmonicolor | Q9UUN9 | 1.00 | 99.9 |
| Tilletiopsis washingtonensis | PWN99435 | 0.34 | 98.8 |
| Rachicladosporium antarcticum | OQO01855 | 0.30 | 97.4 |
| Lodderomyces elongisporus | EDK47269 | 0.28 | 95.9 |
| Acidomyces richmondensis | KYG41666 | 0.26 | 98.4 |
| Plicaturopsis crispa | KII83904 | 0.20 | 97.9 |
| Empty vector (negative control) | n.a. | 0.02-0.05 | n.d. |

n.a. not applicable;
n.d., not determined
UV screening assays were carried out with 3% [v/v] lysate.

0.2 mL-Scale Reactions at 10% [w/w] Substrate Loading Using Lysates

Reactions were carried out in Eppendorf tubes and contained 20 mg of the ketone of formula Ib, 0.1 M potassium phosphate buffer pH 7.2, 2 mM MgCl₂, 0.1% NADP⁺ disodium salt (s/c=100), clarified lysate equivalent to 3.5 mg/mL total protein (determined by the BCA assay) and 8%-20% [v/v]2-propanol. Reactions were run in a thermomixer at 25-30° C. and with shaking at 1,000 rpm. After a given time, reactions were quenched with HPLC-grade methanol to achieve a final dilution factor of 100 and centrifuged. The supernatants were analyzed by HPLC-UV (260 nm). In case of all mutants shown in Tables 22-24, the diastereomeric excess for product the chiral alcohol of formula IIb was ≥99.5%.

TABLE 22
Conversions of the ketone of formula Ib by ketoreductase
mutants at different time points

| | Conversion [a %] with 8% | | |
|---|---|---|---|
| | [v/v] 2-propanol at 28° C. | | |
| Mutations | 2 h | 4 h | 24 h |
| none (wild type) | 2.8 | 4.7 | 18.4 |
| Trp97_Met241_Trp242_Ser245 | 8.3 | 12.6 | 39.6 |
| (E00128) | | | |
| Trp97_Met241_Trp242_Ser245_{—} | 9.9 | 14.2 | 43.1 |
| Met316_Met342 (E00144) | | | |
| Trp97_Lys238_Met241_Trp242_{—} | 16.6 | 24.3 | 70.2 |
| Ser245_Met316_Met342 | | | |
| (E00158) | | | |

TABLE 23
Conversions of the ketone of formula Ib by ketoreductase
mutants at different temperatures

| | Conversion [a %] with 8% | | |
|---|---|---|---|
| | [v/v] 2-propanol after 24 h | | |
| Mutations | 20° C. | 25° C. | 28° C. |
| none (wild type) | 9.1 | 15.6 | 20.0 |
| Trp97_Met241_Trp242_Ser245 | 20.8 | 35.2 | 42.8 |
| (E00128) | | | |
| Trp97_Met241_Trp242_Ser245_{—} | 27.2 | 40.0 | 47.8 |
| Met316_Met342 (E00144) | | | |
| Trp97_Lys238_Met241_Trp242_{—} | 40.7 | 63.3 | 71.2 |
| Ser245_Met316_Met342 | | | |
| (E00158) | | | |

TABLE 24
Conversions of the ketone of formula Ib by ketoreductase
mutants at different 2-propanol concentrations

| | Conversion [a %] at 25° C. after | | |
|---|---|---|---|
| | 24 h 2-propanol concentration | | |
| Mutations | 8% [v/v] | 12% [v/v] | 16% [v/v] |
| none (wild type) | 18.7 | 19.4 | 19.9 |
| Lys238_Asn245 (E00179) | 44.1 | 29.2 | 21.5 |
| Trp97_Lys238_Met241_Trp242_{—} | 51.9 | 49.5 | 25.9 |
| Ser245_Met316_Met342 | | | |
| (E00158) | | | |
| Trp97_Lys238_Met241_Trp242_{—} | 52.2 | 65.5 | 40.2 |
| Ser245_Gly246_Met316_Met342 | | | |
| (E00174) | | | |
| Trp97_Ala224_Lys238_Met241_{—} | 55.5 | 70.2 | 41.6 |
| Trp242_Ser245_Gly246_Met316_{—} | | | |
| Met342 (E00184) | | | |

1 mL-Scale Reactions at 10% [w/w] Substrate Loading Using Lyophilized Lysates

Reactions were carried out in Eppendorf tubes and contained 0.1 g the ketone of formula Ib, 0.1 M potassium phosphate buffer pH 7.2, 2 mM MgCl₂, 0.1% NADP⁺ disodium salt (s/c=100), 8% [v/v]2-propanol and lyophilized lysate derived from selected variants and controls in different substrate-to-enzyme (s/e) ratios. Reactions were run at 23-30° C. and with shaking at 1,500 rpm. After a given time, reactions were quenched with HPLC-grade methanol and analyzed by HPLC-UV (260 nm).

TABLE 25
Conversions and diastereoselectivities of ketoreductase mutants
in 1 mL-scale reactions towards the ketone of formula Ib

| | | Conversion | | |
|---|---|---|---|---|
| | | at 30° C. | | de [%] |
| | | [a %] | | (IIb) |
| Mutations | s/e | 4 h | 24 h | 24 h |
| none | 10 | 5 | 35 | 99.5 |
| Lys238_Asn245 (E00179) | 10 | 25 | 26 | 99.5 |
| Trp97_Met241_Trp242_Ser245 | 5 | 26 | 62* | 99.5* |
| (E00128) | | | | |
| Trp97_Met241_Trp242_Ser245_{—} | 5 | 28 | 58* | 99.5* |
| Met316_Met342 (E00144) | | | | |
| Trp97_Lys238_Met241_Trp242_{—} | 10 | 29 | 53 | 99.5 |
| Ser245_Met316_Met342 (E00158) | | | | |
| Trp97_Lys238_Met241_Trp242_{—} | 10 | 45 | 89 | 99.5 |
| Ser245_Gly246_Met316_Met342 | | | | |
| (E00174) | | | | |
| Trp97_Ala224_Lys238_Met241_{—} | 10 | 29 | 80 | 99.5 |
| Trp242_Ser245_Gly246_Met316_{—} | | | | |
| Met342 (E00184) | | | | |
| Trp97_Thr134_Ala224_Lys238_{—} | 10 | 51 | 96 | 99.5 |
| Met241_Trp242_Ser245_Gly246_{—} | | | | |
| Met316_Met342 (E00185) | | | | |

*20 h

### Example 3: Preparative-Scale Biocatalytic Reaction

20 mL-Scale Reactions at 10% [w/w] Substrate Loading Using 2-Propanol as Final Reductant

Reactions were carried out in a Scott flask with magnetic stirring and contained 2.0 g of the ketone of formula Ib, 0.1 M potassium phosphate buffer pH 7.2, 2 mM MgCl₂, 0.1% NADP⁺ disodium salt (s/c=100), 8% [v/v]2-propanol and lyophilized lysate derived from selected variants and controls in different substrate-to-enzyme (s/e) ratios. The stirred reactions were run at 23-30° C. After a given time, reaction samples (0.05 mL) were quenched with HPLC-grade methanol (0.95 mL) and analyzed by HPLC-UV (260 nm).

TABLE 26
Conversions and diastereoselectivities of ketoreductase
mutants in 20 mL-scale reactions towards the ketone
of formula Ib using 2-propanol as final reductant

| | | Conversion | | |
|---|---|---|---|---|
| | | at 30° C. | | de [%] |
| | | [a %] | | (IIb) |
| Mutations | s/e | 18 h | 46 h | 46 h |
| Trp97_Lys238_Met241_Trp242_Ser245_{—} | 5 | 75 | 93 | 99.5 |
| Met316_Met342 (E00158) | | | | |
| Trp97_Lys238_Gln241_Trp242_Ser245_{—} | 5 | 56 | 66 | 99.5 |
| Met316_Met342 (E00159) | | | | |
| Trp97_Gly238_Met241_Trp242_Ser245_{—} | 5 | 55 | 70 | 99.5 |
| Met316_Met342 (E00160) | | | | |

20 mL-Scale Reactions at 10% [w/w] Substrate Loading Using Glucose as Final Reductant

Reactions were carried out in pH-Stat with overhead stirring and contained 2 g of the ketone of formula Ib, 0.1 M potassium phosphate buffer pH 7.2, 2 mM MgCl₂, 0.1% NADP⁺ disodium salt (s/c=100), 8% [v/v]2-propanol and lyophilized lysate derived from selected variants and controls in different substrate-to-enzyme (s/e) ratios. The stirred reactions were run at 23-30° C. and the pH kept constant by addition of 1 M NaOH. After a given time, reaction samples (0.05 mL) were quenched with HPLC-grade methanol (0.95 mL) and analyzed by HPLC-UV (260 nm).

TABLE 27
Conversions and diastereoselectivities of ketoreductase
mutants in 20 mL-scale reactions towards the ketone
of formula Ib using glucose as final reductant

| | | Conversion | | |
|---|---|---|---|---|
| | | at 30° C. | | de [%] |
| | | [a %] | | (IIb) |
| Mutations | s/e | 2 h | 7 h | 7 h |
| Trp97_Lys238_Met241_Trp242_{—} | 5 | 75 | 100 | 99.5 |
| Ser245_Met316_Met342 (E00158) | | | | |

100 mL-Scale Reaction Using 2-Propanol as Final Reductant

The reaction contained the ketone of formula Ib (10 g, 0.03 mol, 1 eq), water (39 mL), 1 M potassium phosphate buffer pH 7.2 (10 mL), 0.1 M MgCl₂·6H₂O (2 mL), 2-propanol (8 mL) and NADP⁺ (100 mg, 0.004 eq, s/c=100, previously dissolved in 1 mL). After stirring for 5 min, the reaction was started by addition of E00185 lyophilized lysate (2 g, s/e=5, previously dissolved in 30 mL water), followed by incubation at 23° C. and under N₂ flow for 30 h. After the complete reduction (<1.0 a % of the ketone of formula Ib) 2-propanol was depleted by evaporation (40° C., 200-60 mbar), the suspension was cooled to room temperature and the crude product was filtrated, washed with water (twice 25 mL), heptane (twice 25 mL) and dried to constant weight (40° C., <10 mbar). HPLC product purity (as area %, abbreviated as a %) was determined at 254 nm using an Agilent 1290 HPLC system equipped with a Chiralpak IC-3 column (150 mm×4.6 mm, 3 μm) heated at 30° C., and with heptane and ethanol containing 0.1% diethanolamine as solvents A and B, respectively. The flow rate was 0.8 mL/min and the injection volume was 5 μL. The following gradient was used: 0-5 min, B=40%; 5-15 min, B=100%; 15-17 min, B=100%, 17-17.1 min, B: 40%.

9.5 g (94.2%) light beige powder as crude product was isolated with a HPLC purity of 99.7 a % chiral alcohol of formula IIb (0.0 a % (S,S)-trans-product, <0.02 a % cis-products, 0.2 a % of the ketone of formula Ib).

100 mL-Scale Reaction Using Glucose as the Final Reductant

The reaction contained the ketone of formula Ib (10 g, 0.03 mol, 1 eq), water (34 mL), 1 M potassium phosphate buffer pH 7.2 (10 mL), 1 M D(+)-glucose monohydrate (7.13 g, 1.2 eq, 36 mL), 0.1 M MgCl₂·6H₂O (2 mL), 2-propanol (8 mL) and NADP⁺ (100 mg, 0.004 eq, s/c=100) and glucose dehydrogenase GDH-105 from Codexis, Inc. (100 mg, s/e=100). After stirring for 5 min, the reaction was started by addition of E00185 lyophilized lysate (0.2 g, s/e=50). The pH was maintained constant by addition of 1 M NaOH (Tritisol) (28.9 mL, 0.03 mol, 0.96 eq).

The reaction suspension was incubated at 23° C. for 27 h to achieve complete reduction (<1.0 a % of the ketone of formula Ib). Subsequently, the 2-propanol was depleted by evaporation (40° C., 200-60 mbar), the suspension was cooled to room temperature and the crude product was filtrated, washed with water (twice 25 mL), heptane (twice 25 mL) and dried to constant weight (40° C., <10 mbar). HPLC product purity (as area %, abbreviated as a %) was determined as described in the previous section.

9.5 g (94.0%) light beige powder as crude product was isolated with a HPLC purity of 99.5 a % chiral alcohol of formula IIb (0.0 a % (S,S)-trans-product, <0.05 a % cis-products, 0.4 a % of the the ketone of formula Ib).

SEQUENCES

SEQ ID NO: 1
LENGTH: 343
TYPE: Protein
ORGANISM: Sporidiobolus salmonicolor
OTHER INFORMATION: wild type
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSFSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPALALMPP
QYYVSAVDIGLLHLGCLVLPQIERRRVYGTAGTFDWNTVLATFRKLYPSKTFPADFPDQGQ
DLSKFDTAPSLEILKSLGRPGWRSIEESIKDLVGSETA
SEQ ID NO: 2
LENGTH: 343
TYPE: Protein
ORGANISM: Artificial
OTHER INFORMATION: E00128
MUTATIONS: Trp97_Met241_Trp242_Ser245
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPALAMWPP
SYYVSAVDIGLLHLGCLVLPQIERRRVYGTAGTFDWNTVLATFRKLYPSKTFPADFPDQGQ
DLSKFDTAPSLEILKSLGRPGWRSIEESIKDLVGSETA
SEQ ID NO: 3
LENGTH: 343
TYPE: Protein
ORGANISM: Artificial
OTHER INFORMATION: E00144
MUTATIONS: Trp97_Met241_Trp242_Ser245_Met316_Met342
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPALAMWPP
SYYVSAVDIGLLHLGCLVLPQIERRRVYGTAGTFDWNTVLATFRKLYPSKTFPADFPDQGQ
DLSKFDTAPSMEILKSLGRPGWRSIEESIKDLVGSEMA
SEQ ID NO: 4
LENGTH: 343
TYPE: Protein
ORGANISM: Artificial
OTHER INFORMATION: RE1
MUTATIONS: Lys238_Met241_Trp242_Ser245
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSFSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPKLAMWPP
SYYVSAVDIGLLHLGCLVLPQIERRRVYGTAGTFDWNTVLATFRKLYPSKTFPADFPDQGQ
DLSKFDTAPSLEILKSLGRPGWRSIEESIKDLVGSETA
SEQ ID NO: 5
LENGTH: 343
TYPE: Protein
ORGANISM: Artificial
OTHER INFORMATION: E00178
MUTATIONS: Trp97_Lys238_Met241_Trp242_Ser245
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPKLAMWPP
SYYVSAVDIGLLHLGCLVLPQIERRRVYGTAGTFDWNTVLATFRKLYPSKTFPADFPDQGQ
DLSKFDTAPSLEILKSLGRPGWRSIEESIKDLVGSETA
SEQ ID NO: 6
LENGTH: 343
TYPE: Protein
ORGANISM: Artificial
OTHER INFORMATION: E00158
MUTATIONS: Trp97_Lys238_Met241_Trp242_Ser245_Met316_Met342
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPKLAMWPP
SYYVSAVDIGLLHLGCLVLPQIERRRVYGTAGTFDWNTVLATFRKLYPSKTFPADFPDQGQ
DLSKFDTAPSMEILKSLGRPGWRSIEESIKDLVGSEMA
SEQ ID NO: 7
LENGTH: 343
TYPE: Protein
ORGANISM: Artificial
OTHER INFORMATION: E00174
MUTATIONS: Trp97_Lys238_Met241_Trp242_Ser245_Gly246_Met316_
Met342
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTSGWMMSLENGEVSPKLAMWPP
SGYVSAVDIGLLHLGCLVLPQIERRRVYGTAGTFDWNTVLATFRKLYPSKTFPADFPDQGQ
DLSKFDTAPSMEILKSLGRPGWRSIEESIKDLVGSEMA
SEQ ID NO: 8
LENGTH: 343
TYPE: Protein
ORGANISM: Artificial
OTHER INFORMATION: E00184
MUTATIONS: Trp97_Ala224_Lys238_Met241_Trp242_Ser245_Gly246_
Met316_Met342
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSTVSALIPKPNVEGIYLDEKSWNLESIDKAKTLPESDPQKSLWVYAASKTE
AELAAWKFMDENKPHFTLNAVLPNYTIGTIFDPETQSGSTAGWMMSLENGEVSPKLAMWPP
SGYVSAVDIGLLHLGCLVLPQIERRRVYGTAGTFDWNTVLATFRKLYPSKTFPADFPDQGQ
DLSKFDTAPSMEILKSLGRPGWRSIEESIKDLVGSEMA
SEQ ID NO: 9
LENGTH: 343
TYPE: Protein
ORGANISM: Artificial
OTHER INFORMATION: E00185
MUTATIONS: Trp97_Val134_Ala224_Lys238_Met241_Trp242_Ser245_
Gly246_Met316_Met342)
MAKIDNAVLPEGSLVLVTGANGFVASHVVEQLLEHGYKVRGTARSASKLANLQKRWDAKYP
GRFETAVVEDMLKQGAYDEVIKGAAGVAHIASVVSWSNKYDEVVTPAIGGTLNALRAAAAT
PSVKRFVLTSSVVSALIPKPNVEGIYLDEKSWNLESIDKAKTLPESDPQKSLWVYAASKTE
AELAAWKFMDENKPHFTLNAVLPNYTIGTIFDPETQSGSTAGWMMSLENGEVSPKLAMWPP
SGYVSAVDIGLLHLGCLVLPQIERRRVYGTAGTFDWNTVLATFRKLYPSKTFPADFPDQGQ
DLSKFDTAPSMEILKSLGRPGWRSIEESIKDLVGSEMA

1. A mutant ketoreductase with increased ketoreductase activity relative to the wild-type ketoreductase,
wherein the mutant ketoreductase comprises an amino acid sequence that is at least 80% identical to the amino acid sequence of SEQ ID NO: 1 (ketoreductase from Sporidiobolus salmonicolor; UniProt ID: Q9UUN9) and
wherein the mutant ketoreductase has at least one amino acid substitution relative to the amino acid sequence of SEQ ID NO: 1, wherein the amino acid at the position corresponding to position 241 of SEQ ID NO: 1 is substituted.

2. The mutant ketoreductase of claim 1, wherein the amino acid at the position corresponding to position 241 of SEQ ID NO: 1 is substituted with Met (Met241), Asn (Asn241), Arg (Arg241), Trp (Trp241), Ile (241Ile), Lys (Lys241), His (His241), Gln (Gln241), Gly (Gly241), Asp (Asp241), Ser (Ser 241), Thr (Thr241), Tyr (Tyr241), Cys (Cys241), Ala (Ala241), Val (Val241), or Phe (Phe241).

3. The mutant ketoreductase of claim 1, wherein the amino acids at the positions corresponding to positions
241 and 242; or
241 and 245; or
241, 242 and 245
of SEQ ID NO: 1 are substituted.

4. The mutant ketoreductase of claim 1, wherein
the amino acid at the position corresponding to position 241 of SEQ ID NO: 1 is substituted with Met (Met241); and
the amino acid at the position corresponding to position 242 of SEQ ID NO: 1 is substituted with Trp (Trp242); and
the amino acid at the position corresponding to position 245 of SEQ ID NO: 1 is substituted with Ser (Ser245).

5. The mutant ketoreductase of claim 1, wherein
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val 134), Cys (Cys 134), Ala (Ala 134), Gln (Gin134), or Met (Met134) or unsubstituted,
the amino acid at the position corresponding to position 174 of SEQ ID NO: 1 is substituted with Thr (Thr174), Val (Va1174), Met (Met174), Tyr (Tyr174), Ala (Ala174), Ile (Ile174), Lys (Lys174), Arg (Arg174), Asn (Asn174), Ser (Ser174), or Gln (Gln174), or unsubstituted, and/or
the amino acid at the position corresponding to position 224 of SEQ ID NO: 1 is substituted with Ala (Ala224) or unsubstituted,
the amino acid at the position corresponding to position 228 of SEQ ID NO: 1 is substituted with Lys (Lys228), Gln (Gin228), or Arg (Arg228) or unsubstituted,
the amino acid at the position corresponding to position 234 of SEQ ID NO: 1 is substituted with Asp (Asp234), or unsubstituted,
the amino acid at the position corresponding to position 238 of SEQ ID NO: 1 is substituted with Lys (Lys238), Arg (Arg238), Leu (238Leu), Gly (Gly238), His (His238), Asn (Asn238), Trp (Trp238), Asp (Asp238), Thr (Thr238), Ser (Ser238), Gln (Gln238) or Tyr (Tyr238) or unsubstituted,
the amino acid at the position corresponding to position 246 of SEQ ID NO: 1 is substituted with Gly (Gly246), Lys (Lys246), Met (met246) or Ser (246Ser) or unsubstituted,
the amino acid at the position corresponding to position 316 of SEQ ID NO: 1 is substituted with Met (Met316) or unsubstituted, and/or
the amino acid at the position corresponding to position 342 of SEQ ID NO: 1 is substituted with Met (Met342) or unsubstituted.

6. The mutant ketoreductase of claim 1, wherein
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val134) or unsubstituted,
the amino acid at the position corresponding to position 224 of SEQ ID NO: 1 is substituted with Ala (Ala 224) or unsubstituted,
the amino acid at the position corresponding to position 238 of SEQ ID NO: 1 is substituted with Lys (Lys238) or unsubstituted,
the amino acid at the position corresponding to position 241 of SEQ ID NO: 1 is substituted with Met (Met241),
the amino acid at the position corresponding to position 242 of SEQ ID NO: 1 is substituted with Trp (Trp242), and/or
the amino acid at the position corresponding to position 245 of SEQ ID NO: 1 is substituted with Ser (Ser245).

7. The mutant ketoreductase of claim 1, wherein
the amino acid at the position corresponding to position 241 of SEQ ID NO: 1 is substituted with Met (Met241),
the amino acid at the position corresponding to position 242 of SEQ ID NO: 1 is substituted with Trp (Trp242), and
the amino acid at the position corresponding to position 245 of SEQ ID NO: 1 is substituted with Ser (Ser245), and
optionally wherein
the amino acid at the position corresponding to position 97 of SEQ ID NO: 1 is substituted with Trp (Trp97) or unsubstituted,
the amino acid at the position corresponding to position 134 of SEQ ID NO: 1 is substituted with Val (Val134) or unsubstituted,
the amino acid at the position corresponding to position 224 of SEQ ID NO: 1 is substituted with Ala (Ala 224) or unsubstituted,
the amino acid at the position corresponding to position 238 of SEQ ID NO: 1 is substituted with Lys (Lys238) or unsubstituted,
the amino acid at the position corresponding to position 246 of SEQ ID NO: 1 is substituted with Gly (Gly246) or unsubstituted,
the amino acid at the position corresponding to position 316 of SEQ ID NO: 1 is substituted with Met (Met316) or unsubstituted, and/or
the amino acid at the position corresponding to position 342 of SEQ ID NO: 1 is substituted with met (Met342) or unsubstituted.

8. The mutant ketoreductase of claim 1, wherein
the mutant ketoreductase has the mutations Trp97, Met241, Trp242 and Ser245; or
the mutant ketoreductase has the mutations Trp97, Met241, Trp242, Ser245, Met316 and Met342; or
the mutant ketoreductase has the mutations Trp97, Lys238, Met241, Trp242, Ser245, Met316 and Met342; or
the mutant ketoreductase has the mutations Trp97, Lys238, Met241, Trp242, Ser245, Gly246, Met316 and Met342; or
the mutant ketoreductase has the mutations Trp97, 224Ala, Lys238, Met241, Trp242, Ser245, Gly246, Met316 and Met342; or
the mutant ketoreductase has the mutations Trp97, Val134, 224Ala, Lys238, Met241, Trp242, Ser245, Gly246, Met316 and Met342; or
the mutant ketoreductase has the mutations Lys238, Met241, Trp242, Ser245, or
the mutant ketoreductase has the mutations Trp97, Lys238, Met241, Trp242, Ser245.

9. The mutant ketoreductase of claim 1, wherein the mutant ketoreductase consists of or comprises an amino acid sequence that is at least 85%, 90%, 95%, 96%, 97%, 98%, 99%, or 100% identical to the amino acid sequence of any of SEQ ID NO: 2 to 9.

10. The mutant ketoreductase of claim 1, wherein
the ketoreductase activity relative to the wild-type ketoreductase is increased by at least 2.0, 5.0, or 10-fold; and/or
the mutant ketoreductase has an increased conversion relative to the wild-type ketoreductase at 2 to 10% [w/w] substrate loading and at a mutant or wild-type ketoreductase loading of 1-2% [w/w](s/e 5-10) using 2-propanol recycling system; and/or
the mutant ketoreductase is capable of converting a ketone into a chiral alcohol.

11. The mutant ketoreductase of claim 10, wherein the ketone has the formula I 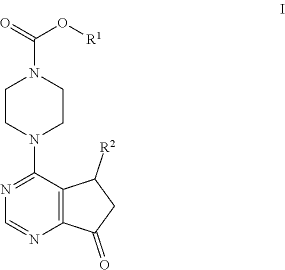
wherein R¹ is C₁₋₄-alkyl and R² is hydrogen or C₁₋₄-alkyl and the resulting chiral alcohol has the formula II 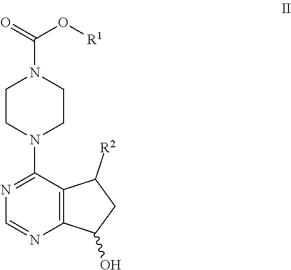
wherein R¹ and R² are as above.

12. The mutant ketoreductase of claim 11, wherein the mutant ketoreductase has the potential to convert the ketone of formula (II) into the corresponding R-enantiomer or R-diastereomer of the chiral alcohol of formula (I) with an enantiomeric or diasteromeric excess of at least 95%, 96%, 97%, 98% or 99%.

13. A nucleic acid coding for the mutant ketoreductase of claim 1, optionally comprised in a vector.

14. A method for the enzymatic reduction of a ketone and the formation of a chiral alcohol in the presence of mutant ketoreductase of claim 1.

15. The method of claim 14, wherein the ketone has the formula I 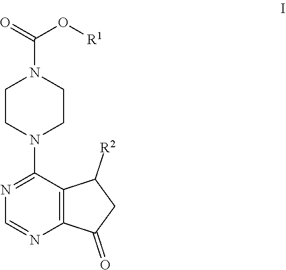
wherein R¹ is C₁₋₄-alkyl and R² is hydrogen or C₁₋₄-alkyl and the resulting chiral alcohol has the formula II 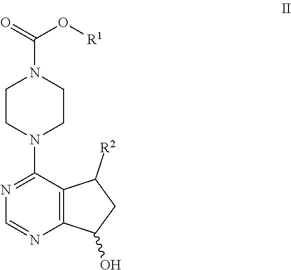
wherein R¹ and R² are as above.

16. The method of claim 15, wherein the resulting chiral alcohol is the corresponding R-enantiomer or R-diastereomer.

17. The method of claim 14 further comprising preparing a serine/threonine protein kinase inhibitor of the formula 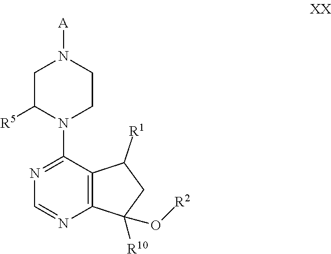
wherein A, R¹, R², R⁵ and R¹⁰ are as defined in claim 1 of the PCT International Application WO 2008/006040 A1
from the chiral alcohol of formula II.

18. The method of claim 14, further comprising preparing a serine/threonine protein kinase inhibitor of the formula 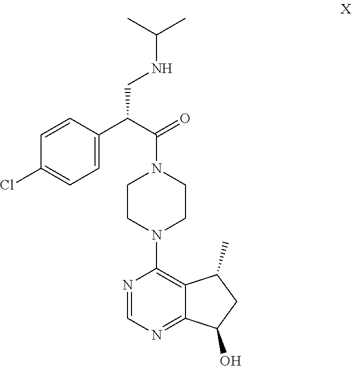 [SMILES: CC(C)NC[C@H](C(=O)N1CCN(c2ncnc3c2[C@H](C)C[C@H]3O)CC1)c1ccc(Cl)cc1]
from the chiral alcohol of formula II.

19. The mutant ketoreductase of claim 3, wherein
the amino acid at the position corresponding to position 241 of SEQ ID NO. 1 is substituted with Met (Met241), Asn (Asn241), Arg (Arg241), Trp (Trp241), Ile (241Ile), Lys (Lys241), His (His241), Gln (Gln241), Gly (Gly241), Asp (Asp241), Ser (Ser 241), Thr (Thr241), Tyr (Tyr241), Cys (Cys241), Ala (Ala241), Val (Val241), or Phe (Phe241); and/or
the amino acid at the position corresponding to position 242 of SEQ ID NO: 1 is substituted with Trp (Trp242), Phe (Phe242), Ile (Ile242), Tyr (Tyr242); Cys (Cys242); Val (Val242), Leu (Leu242), Pro (Pro242), Ala (Ala242), Gln (242Gln) or Ser (Ser242); and/or
the amino acid at the position corresponding to position 245 of SEQ ID NO: 1 is substituted with Ser (Ser245), Thr (Thr245), Asn (Asn245), Met (Met245), Asp (Asp245), Trp (Trp245), Phe (Phe245), Glu (Glu245), Cys (Cys245), or His (His245).


SEQUENCE LISTING (9 sequences)

SEQ ID NO: 1 - protein, 343 aa, Sporidiobolus salmonicolor
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 2 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALAMWPPSYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 3 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALAMWPPSYYVSAVDIGLLHLGCLVLPQIERRRVYGTAGTFDWNTVLATFRKLYPSKTFPADFPDQGQDLSKFDTAPSMEILKSLGRPGWRSIEESIKDLVGSEMA

SEQ ID NO: 4 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPKLAMWPPSYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 5 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPKLAMWPPSYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 6 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPKLAMWPPSYYVSAVDIGLLHLGCLVLPQIERRRVYGTAGTFDWNTVLATFRKLYPSKTFPADFPDQGQDLSKFDTAPSMEILKSLGRPGWRSIEESIKDLVGSEMA

SEQ ID NO: 7 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPKLAMWPPSGYVSAVDIGLLHLGCLVLPQIERRRVYGTAGTFDWNTVLATFRKLYPSKTFPADFPDQGQDLSKFDTAPSMEILKSLGRPGWRSIEESIKDLVGSEMA

SEQ ID NO: 8 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTAGWMMSLFNGEVSPKLAMWPPSGYVSAVDIGLLHLGCLVLPQIERRRVYGTAGTFDWNTVLATFRKLYPSKTFPADFPDQGQDLSKFDTAPSMEILKSLGRPGWRSIEESIKDLVGSEMA

SEQ ID NO: 9 - protein, 343 aa, synthetic construct
MAKIDNAVLPEGSLVLVTGANGFVASHVVEQLLEHGYKVRGTARSASKLANLQKRWDAKYPGRFETAVVEDMLKQGAYDEVIKGAAGVAHIASVVSWSNKYDEVVTPAIGGTLNALRAAAATPSVKRFVLTSSVVSALIPKPNVEGIYLDEKSWNLESIDKAKTLPESDPQKSLWVYAASKTEAELAAWKFMDENKPHFTLNAVLPNYTIGTIFDPETQSGSTAGWMMSLFNGEVSPKLAMWPPSGYVSAVDIGLLHLGCLVLPQIERRRVYGTAGTFDWNTVLATFRKLYPSKTFPADFPDQGQDLSKFDTAPSMEILKSLGRPGWRSIEESIKDLVGSEMA